(19) United States
(12) Patent Application Publication
(10) Pub. No.: US 2026/0234678 A1
(43) Pub. Date: Aug. 13, 2026

(54) VARIANT ISPH AND METHOD FOR SYNTHESIZING COMPOUND HAVING, AS STRUCTURAL UNIT, ISOPRENE OBTAINED BY USING SAID VARIANT ENZYME

(71) Applicant: NATIONAL UNIVERSITY CORPORATION CHIBA UNIVERSITY, Chiba (JP)

(72) Inventor: Daisuke UMENO, Chiba (JP)

(73) Assignee: NATIONAL UNIVERSITY CORPORATION CHIBA UNIVERSITY, Chiba (JP)

(21) Appl. No.: 18/036,585

(22) PCT Filed: Nov. 12, 2021
(86) PCT No.: PCT/JP2021/041812

§ 371 Date: Jul. 24, 2025

(30) Foreign Application Priority Data
Nov. 13, 2020 (JP) 2020-189676

(51) Int. Cl. C12P 9/00; C12N 9/02; C12N 15/10

(52) U.S. Cl. CPC C12P 9/00; C12N 9/0093; C12N 15/1072; C12Y 117/01002

(57) ABSTRACT

Provided are a method for synthesizing a hydrocarbon having, as a structural unit, an isoprene by clarifying an enzyme as a rate-limiting factor for improving the production level of an isoprenoid in an MEP pathway and using a mutant of the enzyme and an MEP pathway from which the rate-limiting factor is eliminated, and a screening system of an enzyme involved in an isoprenoid pathway using an isoprenoid pathway expression system. It has been specified that the enzyme as a rate-limiting factor for improving the production level of the isoprenoid in the MEP pathway is IspH. The present inventors have further confirmed that the production level of isoprenoids is high in the MEP pathway containing a mutant of IspH.

### TECHNICAL FIELD

The present invention relates to a mutant 4-hydroxy-3-methyl-2-butenyl diphosphate reductase (IspH), a method for synthesizing, using the mutant enzyme, a compound having, as a structural unit, isoprene (in particular, a hydrocarbon having, as a structural unit, isoprene), a system containing the mutant enzyme for synthesizing the compound having, as a structural unit, isoprene (in particular, a system for synthesizing the hydrocarbon having, as a structural unit, isoprene), and a method for screening an enzyme involved in an isoprenoid pathway in an isoprenoid pathway expression system with an enhanced enzyme activity for geraniol.

The present application claims the priority of Japanese Patent Application No. 2020-189676, which is incorporated herein by reference.

### BACKGROUND ART

There are 70,000 compounds formed of isoprene units, such as terpenes, terpenoids, and carotenoids, and most of them are products having market value, such as medicines, pigments, flavor or fragrance substances, health foods, cosmetics, resins, and rubber raw materials. Many researchers are aiming at establishment of a highly efficient and stable microorganism production system, and there are many companies that perform metabolism engineering using terpenes as a target.

Improvement in a production amount of terpenes is mainly achieved by an MEV pathway (see: the left side of FIG. 37). It has been found from the study about 15 years that the MEV pathway can be changed to a strengthened pathway having a very high titer by selection of enzymes and expression balance. As the production amount, a production amount of 30 g/L to 100 g/L may also be achieved.

On the other hand, although many studies have been conducted on an MEP pathway (see: the right side of FIG. 37), unlike the MEV pathway, the production amount is not successfully improved.

In a plurality of prior studies, it has not been clarified that which one of enzymes involved in the MEP pathway contributes to a rate-limiting step of the MEP pathway (see: Non-Patent Literatures 1 to 3).

In addition, an effect of increasing the production of carotenoids could not have been confirmed even when all of the seven genes constituting the MEP were overexpressed using a multi-copy plasmid (see: Non-Patent Literatures 4 to 6).

There are reports relating to IspH mutants. However, the MEV pathway containing the IspH mutant cannot contribute to improvement in a production level of α-Pinene (see: Non-Patent Literature 7).

### CITATION LIST

### Patent Literature

Non-Patent Literature 1: Kajiwara et al., Biochem. J., 324, 421-6(1997)
Non-Patent Literature 2: Albrecht et al., Biotechnol. Lett., 21, 791-795 (1999)
Non-Patent Literature 3: Li et al., Metab. Eng., 44, 13-21 (2017)
Non-Patent Literature 4: Yuan et al., Metab. Eng., 8, 79-90 (2006)
Non-Patent Literature 5: Zurbriggen et al., Bioenergy Res., 5, 814-828 (2012)
Non-Patent Literature 6: Zhou et al., J. Biotechnol., 248, 1-8 (2017)
Non-Patent Literature 7: Puan et al., FEBS Lett., 579, 3802-3806 (2005)

### SUMMARY OF INVENTION

### Technical Problem

An object of the present invention is to provide a method for synthesizing a compound having, as a structural unit, isoprene (in particular, a hydrocarbon having, as a structural unit, isoprene), by clarifying an enzyme as a rate-limiting factor for improving the production level of an isoprenoid in an MEP pathway and using a mutant of the enzyme and an MEP pathway from which the rate-limiting factor is eliminated, and a screening system of an enzyme involved in an isoprenoid pathway, which uses an isoprenoid pathway expression system.

### Solution to Problem

As a result of diligent studies, the present inventors have specified that an enzyme that is a rate-limiting factor for improving the production level of isoprenoids in the MEP pathway is IspH. In addition, the present inventors have confirmed that the production level of isoprenoids is high in the MEP pathway containing a mutant of IspH.

Accordingly, the present invention has been completed.

That is, the present invention is as follows.

1. A mutant-IspH gene including any one gene of the following (1) to (8):
(1) a gene encoding a polypeptide consisting of the amino acid sequence shown by SEQ ID NO: 5, 7, 9, or 11;
(2) a gene encoding a polypeptide in which 1 to 30 amino acids are substituted, inserted, deleted, and/or added in the amino acid sequence shown by SEQ ID NO: 5, 7, 9, or 11, and the polypeptide has an activity substantially equivalent to the activity of the polypeptide consisting of the amino acid sequence shown by SEQ ID NO: 5, 7, 9, or 11 of generating IPP (isopentenyl diphosphate) and/or DMAPP (dimethylallyl diphosphate) by using HMB-PP {(E)-4-hydroxy-3-methyl-2-butenyl diphosphate} as a substrate;
(3) a gene encoding a polypeptide which has an identity of 80% or more with the amino acid sequence shown by SEQ ID NO: 5, 7, 9, or 11, and which has an activity substantially equivalent to an activity of the polypeptide consisting of the amino acid sequence shown by SEQ ID NO: 5, 7, 9, or 11 of generating IPP and/or DMAPP by using HMB-PP as a substrate;
(4) a gene consisting of a DNA consisting of the nucleotide sequence shown by SEQ ID NO: 6, 8, 10, or 12;
(5) a gene which hybridizes with a DNA consisting of a nucleotide sequence complementary to the nucleotide sequence shown by SEQ ID NO: 6, 8, 10, or 12 under a stringent condition, and which encodes a polypeptide having an activity substantially equivalent to the activity of the polypeptide consisting of the amino acid sequence shown by SEQ ID NO: 5, 7, 9, or 11 of generating IPP and/or DMAPP by using HMB-PP as a substrate; (6) a gene consisting of a DNA in which 1 to 50 nucleotide sequences are substituted, inserted, deleted, and/or added in the DNA consisting of the nucleotide sequence shown by SEQ ID NO: 6, 8, 10, or 12;
(7) a gene consisting of a DNA having an identity of 80% or more with the DNA consisting of the nucleotide sequence shown by SEQ ID NO: 6, 8, 10, or 12; and
(8) a gene consisting of a DNA consisting of a condensation isomer of the nucleotide sequence shown by SEQ ID NO: 6, 8, 10, or 12.

2. The mutant-IspH gene according to the above item 1, in which the gene is a gene encoding a polypeptide consisting of the amino acid sequence shown by SEQ ID NO: 5 or a gene consisting of a DNA consisting of the nucleotide sequence shown by SEQ ID NO: 6.

3. The mutant-IspH gene according to the above item 1, in which the gene is a gene encoding a polypeptide consisting of the amino acid sequence shown by SEQ ID NO: 7 or a gene consisting of a DNA consisting of the nucleotide sequence shown by SEQ ID NO: 8.

4. The mutant-IspH gene according to the above item 1, in which the gene is a gene encoding a polypeptide consisting of the amino acid sequence shown by SEQ ID NO: 9 or a gene consisting of a DNA consisting of the nucleotide sequence shown by SEQ ID NO: 10.

5. The mutant-IspH gene according to the above item 1, in which the gene is a gene encoding a polypeptide consisting of the amino acid sequence shown by SEQ ID NO: 11 or a gene consisting of a DNA consisting of the nucleotide sequence shown by SEQ ID NO: 12.

6. A method for synthesizing a compound having, as a structural unit, isoprene, the method including: using a cell into which a gene of a mutant IspH is introduced, in which the cell has a high activity for synthesizing isopentenyl diphosphate and/or dimethylallyl diphosphate.

7. The method for synthesizing a compound according to the above 6, in which the mutant-IspH gene is the mutant-IspH gene according to any one of claims 1 to 5.

8. A mutant IspH including any one peptide of the following (1) to (7):
(1) a polypeptide consisting of the amino acid sequence shown by SEQ ID NO: 5, 7, 9, or 11;
(2) a polypeptide which consists of an amino acid sequence in which 1 to 30 amino acids are substituted, deleted, inserted, and/or added in the amino acid sequence shown by SEQ ID NO: 5, 7, 9, or 11, and which has an activity substantially equivalent to an activity of the polypeptide consisting of the amino acid sequence shown by SEQ ID NO: 5, 7, 9, or 11 of generating IPP and/or DMAPP by using HMB-PP as a substrate;
(3) a polypeptide which consists of an amino acid sequence having an identity of 80% or more with the amino acid sequence shown by SEQ ID NO: 5, 7, 9, or 11, and which has an activity substantially equivalent to the activity of the polypeptide consisting of the amino acid sequence shown by SEQ ID NO: 5, 7, 9, or 11 of generating IPP and/or DMAPP by using HMB-PP as a substrate;
(4) a peptide consisting of an amino acid sequence encoded by the nucleotide sequence shown by SEQ ID NO: 6, 8, 10, or 12;
(5) a peptide which consists of an amino acid sequence encoded by a DNA which hybridizes with a DNA consisting of a nucleotide sequence complementary to the nucleotide sequence shown by SEQ ID NO: 6, 8, 10, or 12 under a stringent condition, and which has an activity substantially equivalent to the activity of the polypeptide consisting of the amino acid sequence shown by SEQ ID NO: 5, 7, 9, or 11 of generating IPP and/or DMAPP by using HMB-PP as a substrate;
(6) a peptide consisting of an amino acid sequence encoded by a DNA in which 1 to 50 nucleotide sequences are substituted, deleted, inserted, and/or added in the DNA consisting of the nucleotide sequence shown by SEQ ID NO: 6, 8, 10, or 12; and
(7) a peptide consisting of an amino acid sequence encoded by a DNA having an identity of 80% or more with the DNA consisting of the nucleotide sequence shown by SEQ ID NO: 6, 8, 10, or 12.

9. A mutant IspH or a derivative thereof containing a polypeptide consisting of an amino acid sequence which has amino acids represented by SEQ ID NO: 1 and has an amino acid substitution at 1, 2, 3, 4, 5, 6, or 7 sites selected from the group consisting of the 105th position, the 175th position, the 207th position, the 211th position, the 220th position, the 301st position, and the 306th position.

10. The mutant IspH or the derivative thereof according to the above item 9, in which the mutant IspH has a higher activity for synthesizing isopentenyl diphosphate and/or dimethylallyl diphosphate than wild-type IspH.

11. The mutant IspH or the derivative thereof according to the above item 9 or 10, in which the amino acid substitution is at least one amino acid substitution selected from the following group:
Leu105Gln, Thr175Ser, Thr207Ser, Gln211Leu, Gln220Arg, Glu301Asp, and Asn306Asp.

12. The mutant IspH or the derivative thereof according to any one of the above items 9 to 11, in which the amino acid substitution is selected from any one or more of the following (1) to (4):
(1) Thr207Ser and/or Glu301Asp;
(2) Thr175Ser;
(3) Leu105Gln; and
(4) Gln211Leu, Gln220Arg, and/or Asn306Asp.

13. A mutant IspH or a derivative thereof containing a polypeptide having an amino acid sequence shown by SEQ ID NO: 1 and further having an amino acid substitution that is Thr207Ser and/or Glu301Asp.

14. A mutant IspH or a derivative thereof containing a polypeptide having an amino acid sequence shown by SEQ ID NO: 1 and further having an amino acid substitution that is Thr175Ser.

15. A mutant IspH or a derivative thereof containing a polypeptide having an amino acid sequence shown by SEQ ID NO: 1 and further having an amino acid substitution that is Leu105Gln.

16. A mutant IspH or a derivative thereof containing a polypeptide having an amino acid sequence shown by SEQ ID NO: 1 and further having amino acid substitutions that are Gln211Leu, Gln220Arg and/or Asn306Asp.

17. A method for screening a mutant enzyme involved in an isoprenoid pathway, the method including: introducing a gene library of an enzyme involved in an isoprenoid pathway into a cell with an enhanced enzyme activity for geraniol, a mutation being introduced into the gene library; and using an increase, a decrease, or a loss in a cell growth rate of the cell as an index.

18. The screening method according to the above item 17, in which the increase in the cell growth rate of the cell is an increase in the production level of α-pinene and/or diaponeurosporene.

19. The screening method according to the above item 17 or 18, in which the cell is Escherichia coli.

### Advantageous Effects of Invention

The present invention can provide a mutant IspH by which a production level of an isoprenoid is higher than that of a wild-type IspH, and a method for synthesizing isoprenoid method using the mutant IspH.

### BRIEF DESCRIPTION OF DRAWINGS

FIG. 1 is a plasmid map of pUCara-(40,000)-GESM₅₃his.

FIG. 2 is a plasmid map of pUCara-(40,000)-GESwt.

FIG. 3 is a plasmid map of pACmod-P_{lac}-idi.

FIG. 4 shows results showing an effect of inhibiting growth of Escherichia coli by the over-expression of a geraniol synthase (GES) gene and the elimination thereof.

FIG. 5 is a plasmid map of pAC-mod.

FIG. 6 is a plasmid map of p15A-dxs.

FIG. 7 is a plasmid map of p15A-dxr.

FIG. 8 is a plasmid map of p15A-ispD.

FIG. 9 is a plasmid map of p15A-ispE.

FIG. 10 is a plasmid map of p15A-ispF.

FIG. 11 is a plasmid map of p15A-ispG.

FIG. 12 is a plasmid map of p15A-ispHwt.

FIG. 13 is a plasmid map of p15A-idi.

FIG. 14 is a plasmid map of p15A-ispA.

FIG. 15 (a) of FIG. 15 shows a confirmation of rescue effect from GES-induced toxicity (isoprenoid raw material depletion toxicity) caused by additional expression of each of the MEP enzymes. (b) of FIG. 15 shows results of an effect of an increase in carotenoid production caused by expression of each MEP enzyme. (c) of FIG. 15 is a scheme diagram of a biosynthetic pathway to diaponeurosporene.

FIG. 16 is a schematic diagram of a method for obtaining an improved mutant based on preparation of a random library of IspH and the selection principle of avoiding toxicity of GES expression.

FIG. 17 is a plasmid map of p15A-ispHₘᵤₜ₁.

FIG. 18 is a plasmid map of p15A-ispHₘᵤₜ₂.

FIG. 19 is a plasmid map of p15A-ispHₘᵤₜ₁₂.

FIG. 20 is a plasmid map of p15A-ispHₘᵤₜ₁₄.

FIG. 21 is a plasmid map of pUC18 nm-CrtM_{W38A}-FDS_{Y81A}.

FIG. 22 (a) of FIG. 22 shows a biosynthetic pathway of α-pinene and diaponeurosporene, and (b) of FIG. 22 shows results of production levels of α-pinene and diaponeurosporene.

FIG. 23 is a plasmid map of pJ404-PShis-GPPS.

FIG. 24 is a plasmid map of pUC-crtM-crtN.

FIG. 25 is a plasmid map of pUC18 nm-CrtM_{W38A}-FDS_{Y81A}.

FIG. 26 shows results of a carotenoid synthesis pathway using an IspH mutant and a production level of carotenoids.

FIG. 27 shows the effect of co-expressing IspH mutant on the production level of pinene that is an isoprenoid via non-mevalonate pathway using an IspH mutant.

FIG. 28 is a plasmid map of pJ404-PtPS_{Q47L}-GPPS.

FIG. 29 is a plasmid map of p15A-ispH_{wt}.

FIG. 30 is a plasmid map of p15A-ispH_{wt}-dxs.

FIG. 31 is a plasmid map of p15A-ispH_{wt}-dxs-ispG.

FIG. 32 is a plasmid map of p15A-ispH_{wt}-dxs-ispG-dxr.

FIG. 33 is a plasmid map of p15A-P_{lac}/lacO-ispHₘᵤₜ₁₂.

FIG. 34 is a plasmid map of p15A-ispHₘᵤₜ₁₂-dxs.

FIG. 35 is a plasmid map of p15A-ispHₘᵤₜ₁₂-dxs-ispG.

FIG. 36 is a plasmid map of p15A-ispHₘᵤₜ₁₂-dxs-ispG-dxr.

FIG. 37 is a schematic diagram of an MEP pathway and an MEV pathway.

### DESCRIPTION OF EMBODIMENTS

### (Target of Present Invention)

The present invention relates to a “mutant IspH”, a “method for synthesizing, using the mutant IspH, a compound having, as a structural unit, isoprene (a hydrocarbon having, as a structural unit, isoprene)”, a “system containing the mutant IspH for synthesizing the compound having, as a structural unit, isoprene (the hydrocarbon having, as a structural unit, isoprene)”, and a “method for screening an enzyme involved in an isoprenoid pathway in an isoprenoid pathway expression system with an enhanced enzyme activity for geraniol”.

The “compound having, as a structural unit, isoprene” in the present invention refers to all compounds having isoprene as a partial structure, and examples thereof include isoprenoids, terpenes, carotenoids, cannabinoids, and coenzyme Q10.

The “hydrocarbon having isoprene as a structural unit” in the present invention is not particularly limited, and examples thereof include isoprenoids, terpenes, and carotenoids.

### (Mutant IspH)

The mutant IspH or a derivative thereof according to the present invention means an enzyme whose amino acid or nucleotide is different by one or more from that of a wild-type IspH.

The wild-type IspH is an enzyme that has been found in many prokaryotes including Escherichia coli, and plants, and is known as an enzyme that generates isopentenyl diphosphate (IPP) and dimethylallyl diphosphate (DMAPP) using (E)-4-hydroxy-3-methyl-2-butenyl diphosphate (HMB-PP) as a substrate.

In particular, the mutant IspH or the derivative thereof according to the present invention preferably generates IPP and/or DMAPP in a higher production level (higher synthesis activity) than the wild-type IspH.

The IspH derivatives according to the present invention are protection derivatives, sugar chain modifiers, acylated derivatives, or acetylated derivatives of the mutant IspH.

The protection derivatives, the sugar chain modifiers, the acylated derivatives, or the acetylated derivatives can be obtained by a known method.

The mutant according to the present invention is preferably a mutant having a high enzyme activity as an expression enzyme, expression level, or stability. A mutant capable of increasing substrate specificity is also preferable. In particular, a mutant that enhances the enzyme activity is preferable.

In the method for screening the mutant IspH according to the present invention, a mutant library (gene library in which substitution, deficiency, insertion, and/or addition is introduced into a wild-type IspH gene) is created, and a mutant having a high enzyme activity, expression level, or stability in a host of the mutant can be obtained from the library.

The origin of the gene of the mutant IspH is not limited, and examples thereof include a gene extracted from a natural cell, a gene in a created library, a chemically-synthesized gene, and a PCR-amplified gene. The screening method according to the present invention uses, as an index, the substrate supply ability of an enzyme to be expressed, so that an unidentified gene can also be screened.

Introduction of an ispH gene (a gene library in which substitution, deficiency, insertion, and/or addition is introduced into a wild-type ispH gene) into an isoprenoid-synthesizing cell (a cell in which HMB-PP can be synthesized in the MEP pathway) with the enhanced enzyme activity for geraniol can be performed by a common method used in a gene recombination technology. For example, a gene to be screened is inserted into a vector to transform cells.

The mutant IspH or the derivative thereof according to the present invention has the following mutations in the amino acid sequence as compared with IspH (SEQ ID NO: 1) derived from Escherichia coli.

The substitutions occur at the 105th position, the 175th position, the 207th position, the 211th position, the 220th position, the 301st position, and/or the 306th position.

In addition, the IspH according to the present invention has the following mutations in the amino acid sequence as compared with the IspH (SEQ ID NO: 1) derived from Escherichia coli.

Leu105Gln, Thr175Ser, Thr207Ser, Gln211Leu, Gln220Arg, Glu301Asp, and/or Asn306Asp.

The mutant IspH or the derivative thereof according to the present invention has any one of the following mutations in the amino acid sequence as compared with IspH (SEQ ID NO: 1) derived from Escherichia coli.
(1) Thr207Ser and/or Glu301Asp;
(2) Thr175Ser;
(3) Leu105Gln; and
(4) Gln211Leu, Gln220Arg, and/or Asn306Asp

It has been confirmed in the following examples that the mutant IspH according to the present invention has any one or more of the following amino acid mutations as compared with the IspH (SEQ ID NO: 1) derived from Escherichia coli, and thus generates α-pinene and/or diaponeurosporene isoprene in a higher production level than wild-type IspH derived from Escherichia coli.

| | |
|---|---|
| (1) | Thr207Ser and Glu301Asp (SEQ ID NO: 5) |
| (2) | Thr175Ser (SEQ ID NO: 7) |
| (3) | Leu105Gln (SEQ ID NO: 9) |
| (4) | Gln211Leu, Gln220Arg, and Asn306Asp |
| | (SEQ ID NO: 11) |

The expression that the production level of IPP, DMAPP, α-pinene, and/or diaponeurosporene isoprene is high means that, compared with the wild-type IspH (SEQ ID NO: 1) derived from Escherichia coli, the production level may be 1.1 times or more, 1.2 times or more, 1.3 times or more, 1.4 times or more, 1.5 times or more, 2.0 times or more, 2.1 times or more, 2.2 times or more, 2.3 times or more, 2.4 times or more, 2.5 times or more, 2.6 times or more, 2.7 times or more, 2.8 times or more, 2.9 times or more, 3.0 times or more, about 4.0 times (or more), about 5.0 times (or more), about 6.0 times (or more), about 7.0 times (or more), about 8.0 times (or more), about 9.0 times (or more), about 10 times (or more), about 11 times (or more), about 12 times (or more), about 13 times (or more), about 14 times (or more), about 15 times (or more), about 16 times (or more), about 17 times (or more), about 18 times (or more), about 19 times (or more), about 20 times (or more), about 21 times (or more), about 22 times (or more), about 23 times (or more), about 24 times (or more), about 25 times (or more), about 26 times (or more), about 27 times (or more), about 28 times (or more), about 29 times (or more), about 30 times (or more), about 31 times (or more), about 35 times (or more), about 40 times (or more), about 45 times (or more), about 50 times (or more), about 60 times (or more), about 70 times (or more), about 80 times (or more), about 90 times (or more), about 100 times (or more), about 200 times (or more), about 300 times (or more), about 400 times (or more), about 500 times (or more), about 1,000 times (or more), about 5,000 times (or more), or about 10,000 times (or more).

### (Mutant-IspH Gene)

A mutant-IspH gene according to the present invention includes any one or more of the following genes.

(1) A gene encoding a polypeptide consisting of the amino acid sequence shown by SEQ ID NO: 5, 7, 9, or 11.

(2) A gene encoding a polypeptide in which 1 to 30 amino acids are substituted, inserted, deleted, and/or added in the amino acid sequence shown by SEQ ID NO: 5, 7, 9, or 11, and the polypeptide has an activity substantially equivalent to the activity of the polypeptide consisting of the amino acid sequence shown by SEQ ID NO: 5, 7, 9, or 11 of generating IPP (isopentenyl diphosphate) and/or DMAPP (dimethylallyl diphosphate) by using HMB-PP {(E)-4-hydroxy-3-methyl-2-butenyl diphosphate} as a substrate.

(3) A gene encoding a polypeptide which has an identity of 80% or more with the amino acid sequence shown by SEQ ID NO: 5, 7, 9, or 11 and which has an activity substantially equivalent to the activity of the polypeptide consisting of the amino acid sequence shown by SEQ ID NO: 5, 7, 9, or 11 of generating IPP and/or DMAPP by using HMB-PP as a substrate.

(4) A gene consisting of a DNA consisting of the nucleotide sequence shown by SEQ ID NO: 6, 8, 10, or 12.

(5) A gene which is hybridizes with a DNA consisting of a nucleotide sequence complementary to the nucleotide sequence shown by SEQ ID NO: 6, 8, 10, or 12 under a stringent condition, and which encodes a polypeptide having an activity substantially equivalent to the activity of the polypeptide consisting of the amino acid sequence shown by SEQ ID NO: 5, 7, 9, or 11 of generating IPP and/or DMAPP by using HMB-PP as a substrate.

(6) A gene consisting of a DNA in which 1 to 50 nucleotide sequences are substituted, inserted, deleted, and/or added in the DNA consisting of the nucleotide sequence shown by SEQ ID NO: 6, 8, 10, or 12.

(7) A gene consisting of a DNA having an identity of 80% or more with the DNA consisting of the nucleotide sequence shown by SEQ ID NO: 6, 8, 10, or 12.

(8) A gene consisting of a DNA consisting of a condensation isomer of the nucleotide sequence shown by SEQ ID NO: 6, 8, 10, or 12.

The gene in the above (1) is preferably a gene encoding a mutant ispH obtained by the method for screening the mutant IspH according to the present invention. The mutant IspH has an activity of generating IPP and/or DMAPP using HMB-PP as a substrate.

The gene in the above (2) is a gene encoding a polypeptide into which a mutation that does not cause the mutant ispH according to the present invention to lose the enzyme activity is introduced. Such mutations also include artificial mutations in addition to mutations occurring in nature. Examples of methods for generating an artificial mutation include a site-specific mutation induction method (Nucleic Acids Res. 10, 6487 to 6500, 1982). The number of mutated amino acids is normally 30 or less, preferably 20 or less, more preferably 10 or less, still more preferably 5 or less, and most preferably 3 or less. Whether the polypeptide into which the mutation has been introduced retains the enzyme activity can be determined, for example, by introducing a gene encoding the polypeptide, into which the mutation has been introduced, into Escherichia coli or the like, causing the gene to express, and examining whether the Escherichia coli or the like can generate IPP and/or DMAPP by using HMB-PP as a substrate. There is a high possibility that the enzyme activity is lost when a mutation is introduced into positions or sites of these amino acids, so that the mutation is preferably introduced into an amino acid other than these amino acids.

The “activity substantially equivalent to the activity of the polypeptide consisting of the amino acid sequence shown by SEQ ID NO: 5, 7, 9, or 11 of generating IPP and/or DMAPP by using HMB-PP as a substrate” in the above (2), (3), and/or (5) means that the activity degree thereof may be stronger or weaker than the activity of the polypeptide consisting of the amino acid sequence shown by SEQ ID NO: 5, 7, 9, or 11 of generating IPP and/or DMAPP by using HMB-PP as a substrate. Examples of the above activity include the activity by 0.1 times, 0.2 times, 0.3 times, 0.4 times, 0.5 times, 0.6 times, 0.7 times, 0.8 times, 0.9 times, 1.0 times, 1.1 times, 1.2 times, 1.3 times, 1.4 times, 1.5 times, 2.0 times, 2.1 times, 2.2 times, 2.3 times, 2.4 times, 2.5 times, 2.6 times, 2.7 times, 2.8 times, 2.9 times, 3.0 times, 4.0 times, 5.0 times, 10 times, 15 times, 20 times, 30 times, 40 times, 50 times, 100 times, 500 times, or 1,000 times.

The identity (homology) can be calculated using basic local alignment search tool at the national center for biological information (BLAST) or the like (for example, using default, that is, initial setting parameters).

The gene in the above (3) includes a gene encoding a polypeptide having an identity (homology) of 80% or more or 90% or more, preferably 93% or more, more preferably 95% or more, and still more preferably 98% or more, and an identity (homology) of most preferably 99% or more, with the amino acid sequence shown by SEQ ID NO: 5, 7, 9, or 11.

The gene in the above (5) is a gene obtained by using hybridization between DNAs. The “stringent condition” as to the gene means a condition under which only specific hybridization occurs and non-specific hybridization does not occur. Such a condition is normally a condition of hybridization at 37° C. in a buffer solution containing 5×SSC and 1% SDS and a washing treatment at 37° C. with a buffer solution containing 1×SSC and 0.1% SDS, preferably a condition of hybridization at 42° C. in a buffer solution containing 5×SSC and 1% SDS and a washing treatment at 42° C. with a buffer solution containing 0.5×SSC and 0.1% SDS, and more preferably a condition of hybridization at 65° C. in a buffer solution containing 5×SSC and 1% SDS and a washing treatment at 65° C. with a buffer solution containing 0.2×SSC and 0.1% SDS. Whether the DNA obtained by using hybridization encodes a polypeptide having activity can be determined, for example, by introducing the DNA into Escherichia coli or the like, causing the DNA to express, and examining whether the Escherichia coli or the like can produce IPP and/or DMAPP by using HMB-PP as a substrate. The DNA obtained by hybridization normally has a high identity (homology) with the gene (SEQ ID NO: 6, 8, 10, or 12) in the above (4). The high identity (homology) refers to an identity of 80% or more or 90% or more, preferably an identity of 95% or more, more preferably an identity of 98% or more, and most preferably an identity of 99% or more.

The gene in the above (6) is a gene consisting of a DNA in which 1 to 50, preferably 1 to 30, more preferably 1 to 20, particularly preferably 1 to 10, and most preferably 1 to 5 nucleotide sequences are substituted, deleted, inserted, and/or added in the DNA consisting of the nucleotide sequences shown by SEQ ID NO: 6, 8, 10, or 12.

The gene in the above (7) includes a gene consisting of a DNA having an identity of 80% or more or 90% or more, preferably 93% or more, more preferably 95% or more, and still more preferably 98% or more, and an identity of most preferably 99% or more, with the DNA consisting of the nucleotide sequence shown by SEQ ID NO: 6, 8, 10, or 12.

The condensation isomer of the nucleotide sequence in the above (8) means another nucleotide sequence corresponding to the amino acid encoded by the nucleotide sequence shown by SEQ ID NO: 6, 8, 10, or 12.

### (Mutant IspH)

The mutant IspH according to the present invention has any one of the following peptides.

(1) A polypeptide consisting of an amino acid sequence shown by SEQ ID NO: 5, 7, 9, or 11.

(2) A polypeptide which consists of an amino acid sequence in which 1 to 30 or 1 to 20, preferably 1 to 15, more preferably 1 to 10, and most preferably 1 to 5 amino acids are substituted, deleted, inserted, and/or added in the amino acid sequence shown by SEQ ID NO: 5, 7, 9, or 11, and which has an activity substantially equivalent to an activity of the polypeptide consisting of the amino acid sequence shown by SEQ ID NO: 5, 7, 9, or 11 of generating IPP and/or DMAPP by using HMB-PP as a substrate.

(3) A polypeptide which consists of an amino acid sequence having an identity of 80% or more, 85% or more, 90% or more, 93% or more, 95% or more, or 97% or more with the amino acid sequence shown by SEQ ID NO: 5, 7, 9, or 11, and which has an activity substantially equivalent to the activity of the polypeptide consisting of the amino acid sequence shown by SEQ ID NO: 5, 7, 9, or 11 of generating IPP and/or DMAPP by using HMB-PP as a substrate.

(4) A peptide consisting of an amino acid sequence encoded by the nucleotide sequence shown by SEQ ID NO: 6, 8, 10, or 12.

(5) A peptide which consists of an amino acid sequence encoded by a DNA which hybridizes with a DNA consisting of a nucleotide sequence complementary to the nucleotide sequence shown by SEQ ID NO: 6, 8, 10, or 12 under a stringent condition, and which has an activity substantially equivalent to the activity of the polypeptide consisting of the amino acid sequence shown by SEQ ID NO: 5, 7, 9, or 11 of generating IPP and/or DMAPP by using HMB-PP as a substrate.

(6) A peptide consisting of an amino acid sequence encoded by a DNA in which 1 to 50 (1 to 30 or 1 to 20, preferably 1 to 15, more preferably 1 to 10, and most preferably 1 to 5) nucleotide sequences are substituted, deleted, inserted, and/or added in the DNA consisting of the nucleotide sequence shown by SEQ ID NO: 6, 8, 10, or 12.

(7) A peptide consisting of an amino acid sequence encoded by a DNA having an identity of 80% or more (85% or more, 87% or more, 90% or more, 93% or more, 95% or more, or 97% or more) with the DNA consisting of the nucleotide sequence shown by SEQ ID NO: 6, 8, 10, or 12.

From the viewpoint of not changing basic properties (physical properties, functions, a physiological activity, an immunological activity, and the like) of the peptide in the introduction of the mutation in the peptide, mutual substitution between amino acids of the same group (polar amino acids, nonpolar amino acids, hydrophobic amino acids, hydrophilic amino acids, positive charged amino acids, negative charged amino acids, aromatic amino acids, and the like) is easily assumed.

### (Screening Method According to Present Invention)

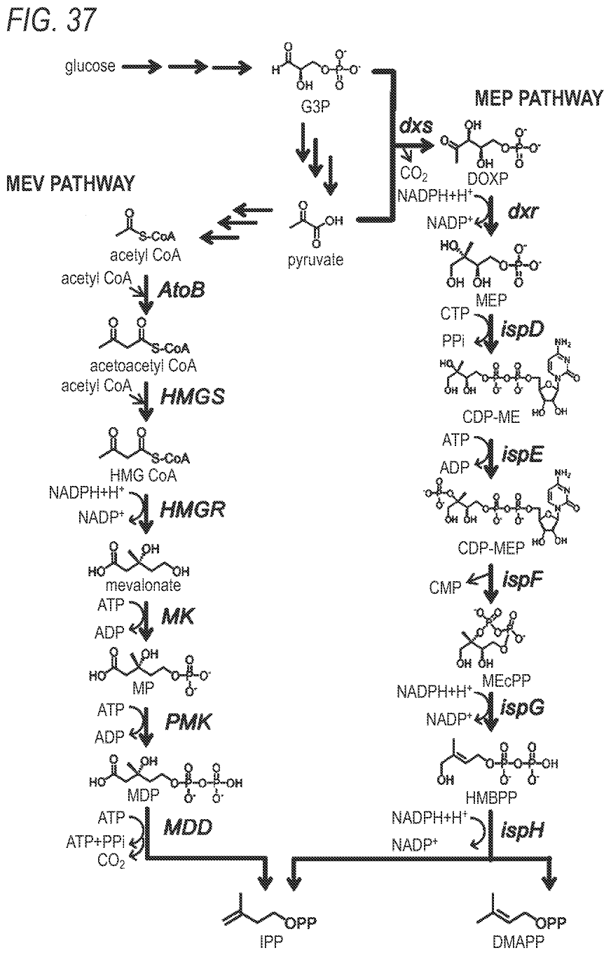

The method according to the present invention for screening an enzyme involved in an isoprenoid pathway in an isoprenoid pathway expression system with an enhanced enzyme activity for geraniol (hereinafter, may be abbreviated as “screening method according to the present invention”) relates to a screening method for searching a mutant enzyme involved in an isoprenoid pathway {for example, an MEP pathway: a DOXP synthase (Dxs), a DOXP reductoisomerase (Dxr, IspC), a 4-diphosphocytidyl-2-C-methyl-D-erythritol synthase (IspD), a 4-diphosphocytidyl-2-C-methyl-D-erythritol kinase (IspE), a 2-C-methyl-D-erythritol-2,4-cyclodiphosphate synthase (IspF), an HMB-PP synthase (IspG), an HMB-PP reductase (IspH), an MEV pathway: an acetyl CoA acetyl transferase, an HMG-CoA synthase, an HMG-CoA reductase, a mevalonate kinase, a 5-phosphomevalonate kinase, and a diphosphomevalonate decarboxylase}. In the screening method, a mutant enzyme gene (preferably an enzyme gene library involved in an isoprenoid pathway, particularly an enzyme gene library which is involved in an isoprenoid pathway and into which a random mutation has been introduced) involved in an isoprenoid pathway was introduced into a cell with an enhanced enzyme activity for geraniol capable of synthesizing products of either the MEV pathway or the MEP pathway shown in FIG. 37, and the survival rate of the introduced cell, the production amount of products in each pathway, and the production amount of geraniol, diaponeurosporene, ubiquinone, bactoprenol, or the like are used as an index.

(Cells with Enhanced Enzyme Activity for Geraniol Capable of Synthesizing Products of Either MEV Pathway or MEP Pathway)

The “cells with an enhanced enzyme activity for geraniol” in the present invention are characterized in that the enzyme activity for geraniol is higher than that of the wild-type cells. Note that the cells having high enzyme activity for geraniol can be obtained by over-expression of geraniol synthase (in particular, geraniol synthase having a high activity) in cells, by a known method.

The “cells capable of synthesizing products of either the MEV pathway or the MEP pathway” in the present invention may contain each enzyme and substrates shown in FIG. 37. The enzymes and the substrates may be endogenous of cells, or may be exogenous due to the introduction of a gene or a protein from the outside.

In addition, the cells according to the present invention may be natural cells or cells transformed by gene recombination. Examples of the genetically recombinant cells include Escherichia coli, Bacillus subtilis, yeast, and the like, which are cells easy for the operation of gene recombination.

### (Compound Having, as Structural Unit, Isoprene by Using Mutant Enzyme Involved in Isoprenoid Pathway)

In a method for synthesizing, using the mutant enzyme involved in the isoprenoid pathway, a compound having, as a structural unit, isoprene, according to the present invention (in particular, a method for synthesizing hydrocarbons having, as a structural unit, isoprene, and further an isoprenoid synthesis method, hereinafter, may be abbreviated as a “synthesis method according to the present invention”), the enzyme involved in the mutant-isoprenoid pathway, which is obtained in the above “screening method according to the present invention” and has a high enzyme activity, is used.

As the synthesis method according to the present invention, a known method can be used except that the mutant enzyme involved in the isoprenoid pathway is used. For example, by introducing a gene of the mutant enzyme involved in the isoprenoid pathway into a cell strain having an MEV pathway and/or an MEP pathway to express the enzyme in the cell strain, products and the like in the MEV pathway and/or the MEP pathway can be expressed with a high yield. Alternatively, an artificial pathway for directly diphosphorylating DMAOH or IOH, a pathway (J M. Clomurg, PNAS, 116, 12810 (2019)), that for supplying IPP/DMAPP from glycerol via IOH/DMAOH, and further a pathway (Perez-Gil, Plos ONE, 8, e43775) for creating DOXP from another pathway by modifying the specificity of an enzyme in another pathway, which have been actively studied recently, can also be used in combination.

It is confirmed that an isoprenoid can be synthesized with a high yield by the synthesis method using the mutant IspH according to the present invention.

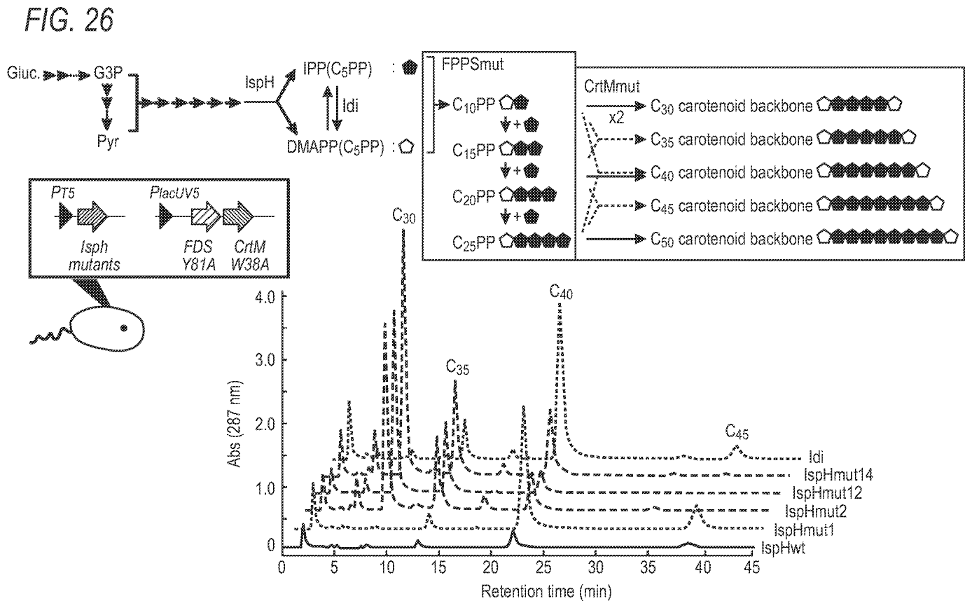

In the synthesis method according to the present invention, the results of the following Examples show that a ratio of the number of carbon atoms in the hydrocarbon containing isoprene as a structural unit can be changed depending on the kind of mutant IspH to be used. For example, as is clear from the results of FIG. 26, IspHₘᵤₜ₁ (SEQ ID NO: 5) is used in order to increase a content ratio of carotenoids having a high carbon number (for example, C45 and C40: carotenoids having a high molecular weight).

In order to increase the content ratio of carotenoids having a low carbon number (for example, C35 and C30: carotenoids having a low molecular weight), IspHₘᵤₜ₂ (SEQ ID NO: 7), IspHₘᵤₜ₁₂ (SEQ ID NO: 9), and IspHₘᵤₜ₁₄ (SEQ ID NO: 11) are used.

In the synthesis method according to the present invention, the results of the following examples show that a ratio of DMAPP to IPP to be synthesized can be adjusted according to the type of mutant IspH to be used.

For example, as is clear from the results of FIG. 26, when IspHₘᵤₜ₁ is used, C45 and C40 can be efficiently synthesized by decreasing the DMAPP/IPP ratio. In addition, when IspH_{mut2,12,14} are used, C35 and C30 can be efficiently synthesized by increasing the DMAPP/IPP ratio.

In the synthesis method having both pathways that are the MEV pathway and the MEP pathway according to the present invention, it is necessary to increase the DMAPP/IPP ratio in order to simultaneously cause both pathways that are the MEV pathway and the MEP pathway to express with high efficiency (see: FIG. 37). That is, in the synthesis method using IspH_{mut2,12,14} according to the present invention, both pathways that are the MEV pathway and the MEP pathway can be simultaneously forced to act with high efficiency by increasing the DMAPP/IPP ratio.

### (Isoprenoid Synthesis System Containing Mutant Enzyme Involved in Isoprenoid Pathway)

A compound (in particular, isoprenoid) synthesis system containing a mutant enzyme involved in the isoprenoid pathway according to the present invention (hereinafter, may be abbreviated as the “synthesis system according to the present invention”) may be subjected to the “synthesis method according to the present invention”.

The synthesis system according to the present invention may use any known composition as long as it contains the mutant enzyme involved in the isoprenoid pathway. For example, the synthesis system according to the present invention includes a cell strain having an MEV pathway and an MEP pathway, and a gene of a mutant enzyme involved in the isoprenoid pathway.

### EXAMPLES

Hereinafter, the present invention will be specifically described with reference to Examples, but the present invention is not limited to these Examples.

### Example 1

### {Construction of Screening System of Enzyme Involved in Isoprenoid Pathway}

In this Example, a screening system for measuring the enzyme activity of the enzyme involved in the isoprenoid pathway was established. Details are as follows.

pUCara-(400000)-GES_{M53} was introduced into BW25113Δtna (deletion of the indole productionenzyme tryptophanase), BW25113, DH5a, and XL1blue that are four different Escherichia coli strains. It was confirmed that the expression of the activity-enhanced GES mutant (GES_{M53}) can be induced at a high yield by adding arabinose to a medium containing these transformants (see: Tashiro et al., ACS Synth. Biol., 5, 1011-1021 (2016)).

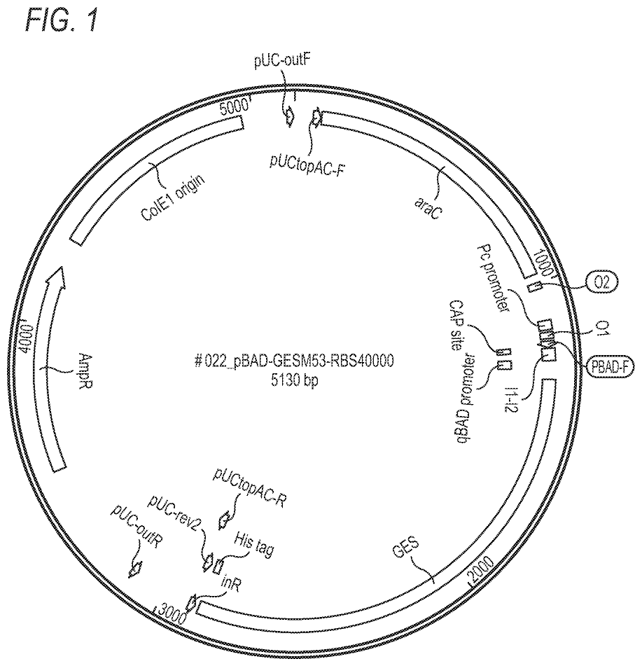

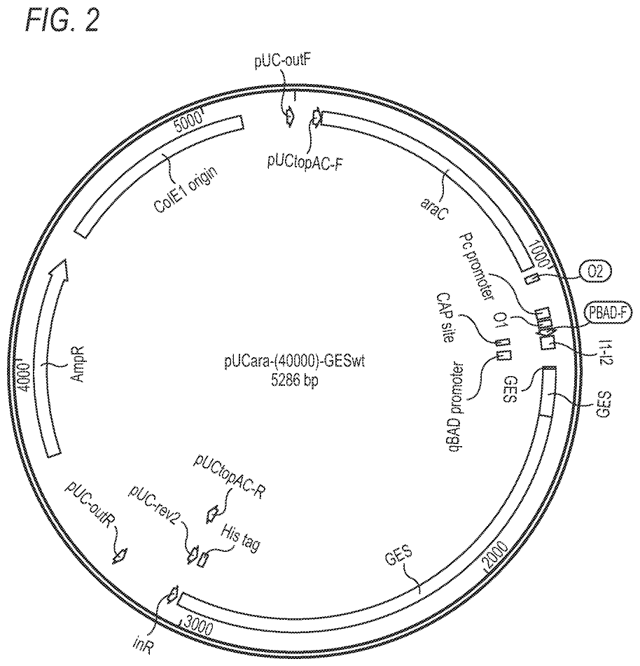

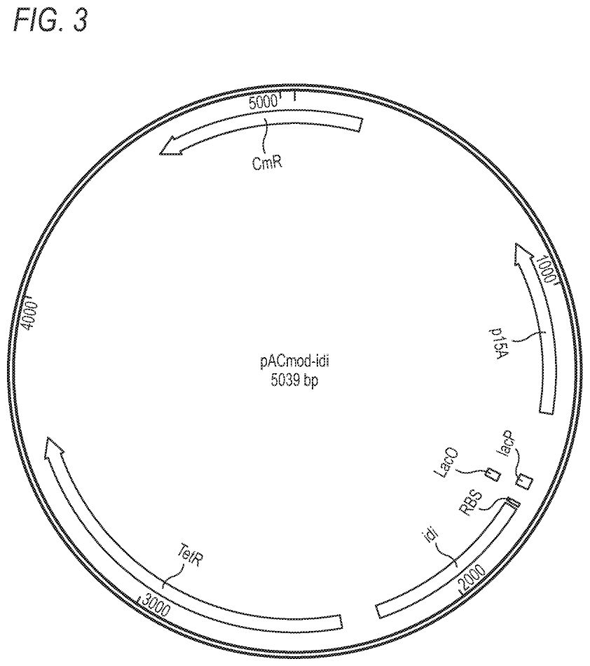

The used pUCara-(40,000)-GES_{M53}his, pUCara-(40,000)-GESwt, and pACmod-P_{lac}-idi are shown in FIGS. 1 to 3, respectively.

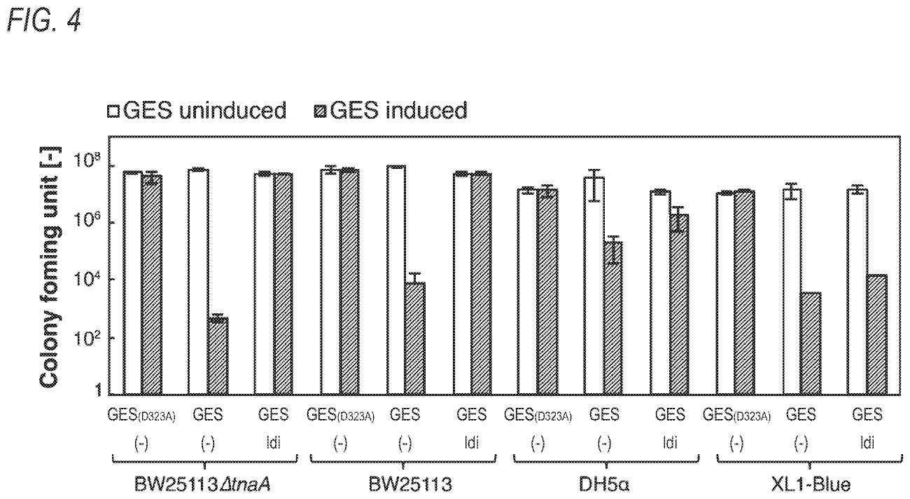

In an LB agar medium supplemented with 0.2% of arabinose, about 1,000,000 cells of each of the four kinds of transformants was spread (under conditions with or without Idi addition) to form colonies. The number of colony-forming units when the expression of GES_{M53} was induced in all the transformants (shown by a “gray bar” in FIG. 4) was significantly smaller than that when the expression was not induced (an agar medium containing no arabinose, shown by a “white bar” in FIG. 4). In the case of DH5a, the number of colony-forming units was decreased by about one digit, and in the case of BW25113(Δtna), the number of colony-forming units was decreased as much as by five digits. In addition, the GES_{M53} mutant (GES (D323A)) into which an inactivated mutation (D323A) was introduced did not change the colony-forming ability for any strain.

Accordingly, it was confirmed that the enzyme activity of GES contributed to the decrease in cfu due to GES expression.

There are two possibilities for cell growth inhibition due to the GES_{M53M53} activity. One possibility is the product toxicity of GES. A monoterpene including geraniol is a substance widely used as an antibacterial agent, and excessive production thereof may inhibit growth of the production strain. The other possibility is the depletion of metabolites due to the GES_{M53} activity. On a metabolism pathway of Escherichia coli, the accumulation amount of geranyl diphosphate (GPP) as a GES precursor is low. GPP is not only used for tRNA modification (geranylation) (see: Dumelin et al., Nat. Chem. Biol., 8, 913-9 (2012)), but also used as a raw material of quinone (an electron transfer mediator of a respiratory chain) or bactoprenol (required for cell wall synthesis), and a precursor of farnesyl diphosphate. In the case where GPP is sucked up by a strong activity of GES_{M53}, cell growth may stop due to depletion of FPP that is essential for growth.

The same experiment was carried out by co-expressing isopentenyl diphosphate isomerase gene (idi) (Kajiwara et al., Biochem. J., 324,421-6(1997)) having an effect of increasing isoprenoid production by about three times, and as a result, in any of the strains, a decrease in colony-forming ability due to induction of GES expression was greatly prevented (see: FIG. 4). As a result, the isoprenoid raw materials (IPP, DMAPP) increased by the Idi expression alleviated the substrate consumption of GES. In the case of the accumulated toxicity of geraniol, the colony-forming ability is to be further reduced.

In the case of XL1blue and DH5a, cfu was unable to completely recover. However, in the cases of BW25113 and BW25113(Δtna) strains, the colony-forming ability decreased by 4 to 5 digits upon GES_{M53} expression, and it completely recovered by the expression of idi alone. It has been reported that a decrease in cell growth due to farnesene synthase (FS) means that a growth rate is decreased by about 2 times (see: Wang et al., Metab. Eng., 13, 648-655 (2011)). On the other hand, in the system according to the present Example, cfu was also decreased by a maximum of five digits due to GES_{M53} expression, and completely recovered by additional expression of idi. That is, by the expression induction of GES_{M53}, the cells expressing idi were concentrated up to 100,000 times overnight as compared with the cells not expressing idi.

As described above, the screening system for the enzyme involved in the isoprenoid pathway, which uses an isoprenoid pathway expression system with an enhanced enzyme activity for geraniol, constructed.

### Example 2

(Overexpression of Enzyme Involved in Isoprenoid Pathway in Isoprenoid Pathway Expression System with Enhanced Enzyme Activity for Geraniol)

In the present Example, the effect of increasing the production of carotenoids due to overexpression of the enzyme involved in the isoprenoid pathway in the isoprenoid pathway expression system with an enhanced enzyme activity for geraniol, which was constructed in Example 1, was confirmed. Details are as follows.

There are seven kinds of enzymes constituting the MEP: DXS, DXR, IspD, IspE, IspF, IspG, and IspH. It was checked how much the additional overexpression of these enzymes had a rescue effect on GES expression toxicity (growth inhibition due to depletion of an isoprenoid raw material).

Specifically, plasmids for causing a total of nine kinds of genes that are genes of various MEP enzymes (dxs, dxr, ispD, ispE, ispF, ispG, ispH), idi, and ispA to singly and constantly express were prepared using a pAC vector as a base. Each of the plasmids was introduced into cells transformed with pUCara(40,000)-GES_{M53}his (BW25113Δtna strains), and a value obtained by dividing the number of colony-forming units under GES_{M53} induction conditions (an agar medium supplemented with 0.2% arabinose) by the number of colonies on a non-induction plate (an agar medium not supplemented with arabinose) was examined. With respect to a strain in which nothing was expressed, the number of colony-forming units was decreased to about 1/10,000 (0.01%) by the addition of arabinose.

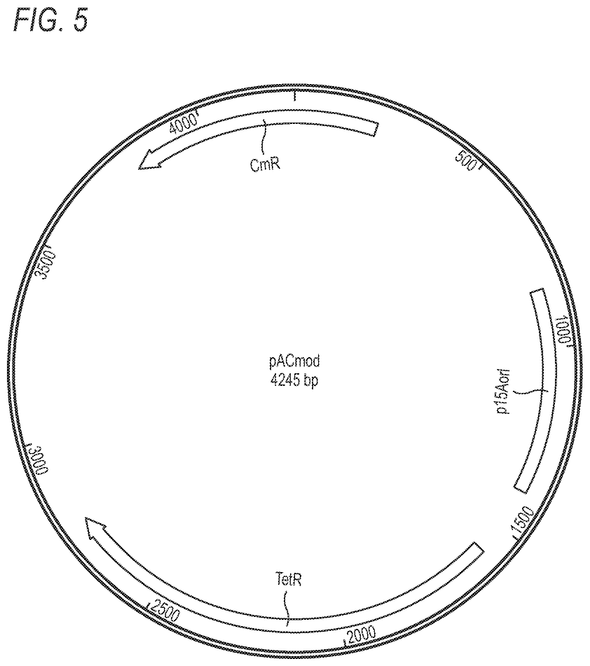

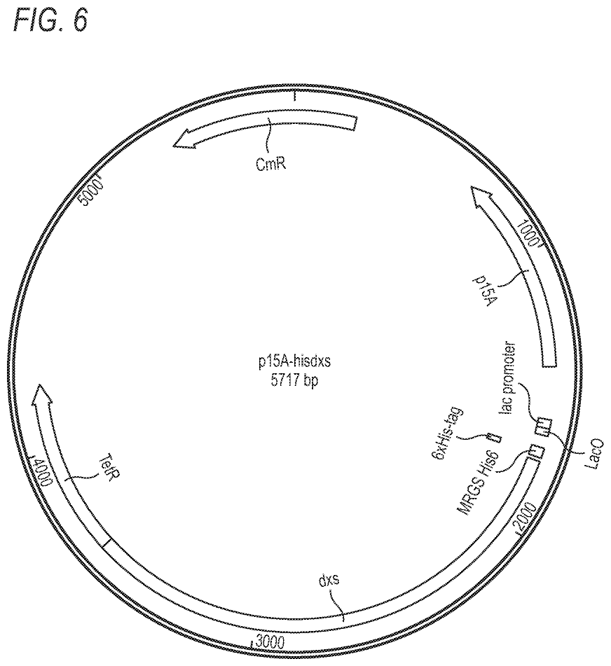

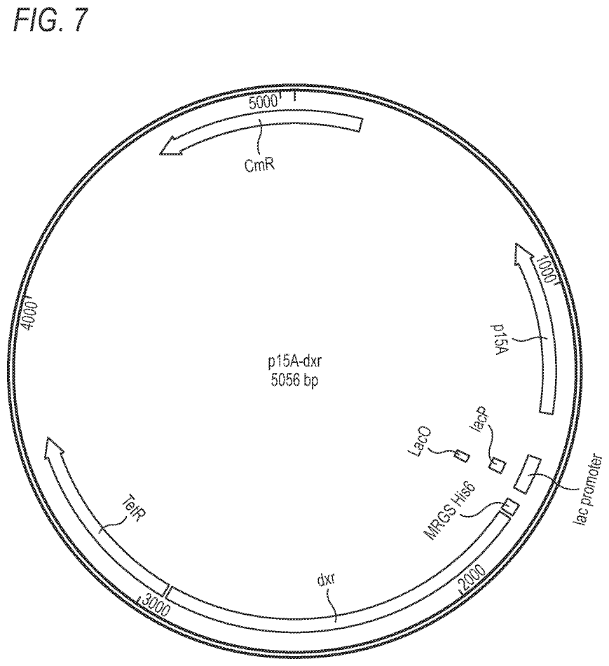

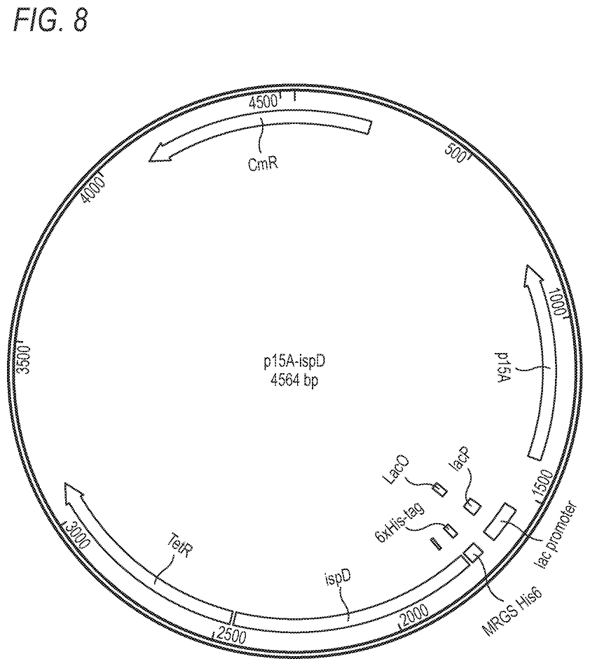

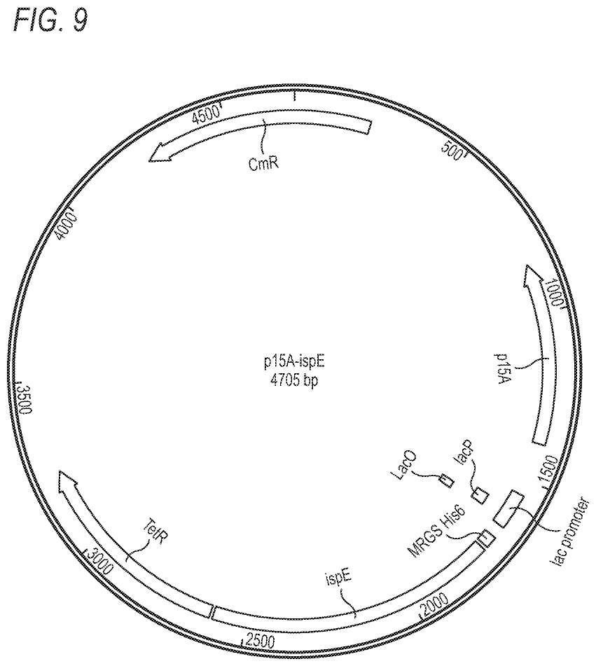

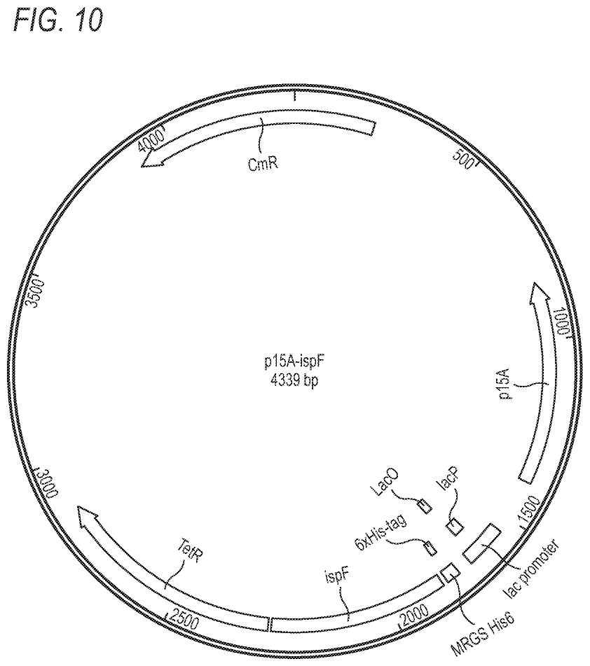

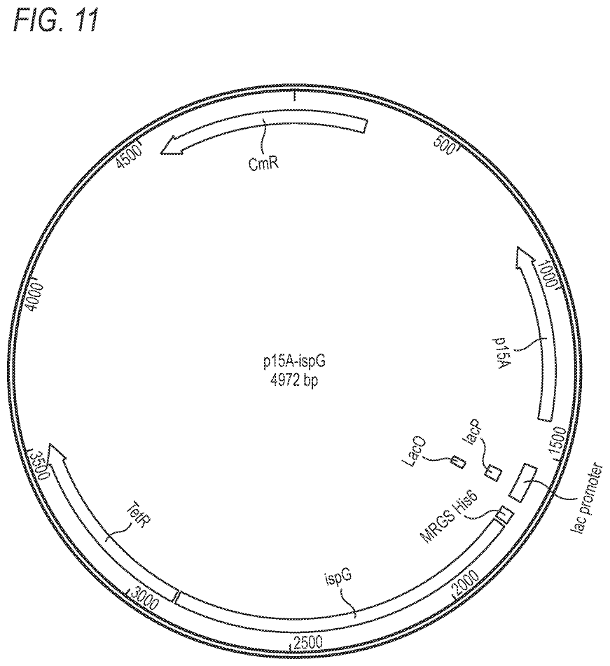

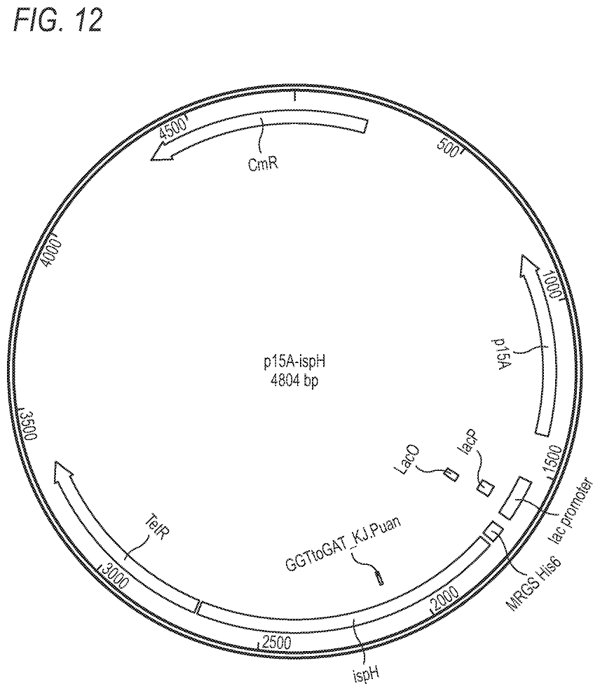

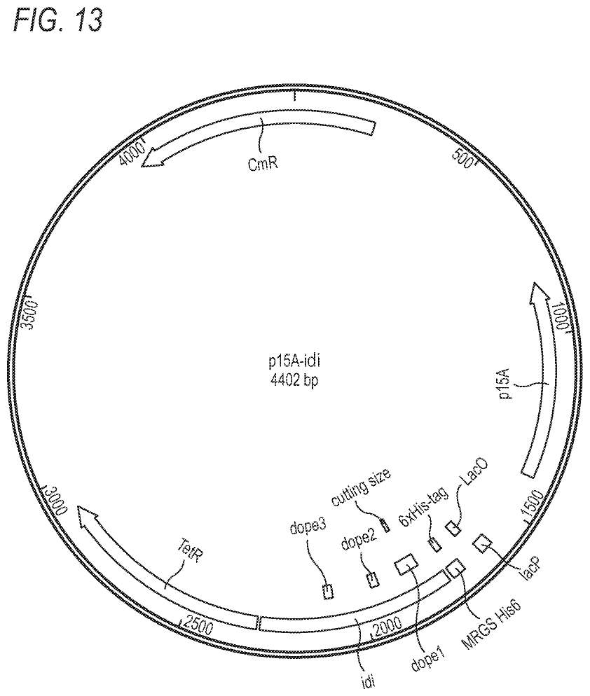

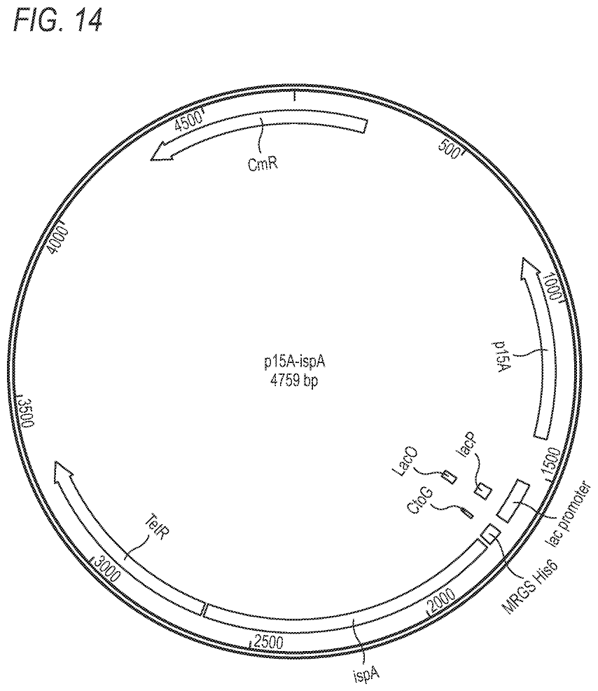

FIGS. 5 to 14 show plasmid maps of nine used plasmids into which pACmod and nine kinds of genes were inserted.

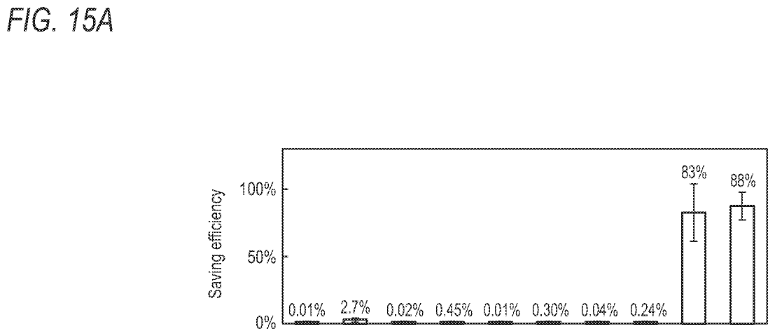

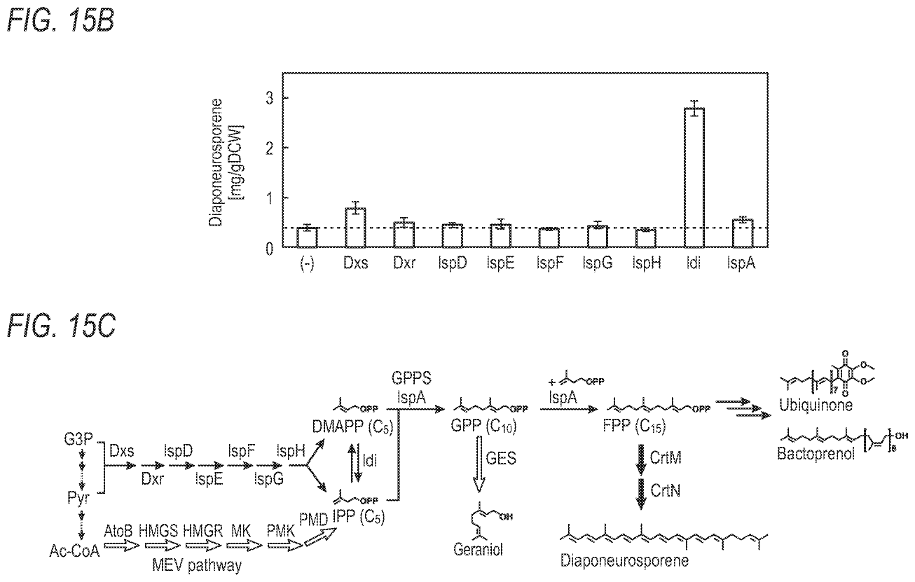

The overexpression of Dxs was confirmed to have a rescue effect of about 270 times (a of FIG. 15: colony-forming rate up to 2.7%), and the overexpression of other MEP enzymes (DXR, IspD, IspE, IspF, IspG, and IspH) had a rescue effect of 1 time to 45 times. It was confirmed that the overexpression of these MEP enzymes was lower than the overexpression of Idi (8,300 times) and the overexpression of IspA (8,800 times) (a of FIG. 15).

In addition, the effects of the expression of these nine genes on the increase in production of carotenoids as an isoprenoid dye were examined. Specifically, each of these single and constant expression plasmids was co-introduced with pUC-MN into the BW25113Δtna strain, and the amount of carotenoids accumulated per cell was measured according to the previously reported procedure (b of FIG. 15). The rescue ability confirmed in a of FIG. 15 showed a good correlation with the isoprenoid raw material supply capacity brought about by the expression of the respective genes. Dxs, which is an MEP enzyme having the highest rescue effect, was observed to have an effect of increasing the biosynthesis amount of diaponeurosporene, which is an isoprenoid dye, by two times (b of FIG. 15). Idi, which provides the rescue effect of 8,300 times, exhibited the production-increasing effect of 6 times or more. Note that. The production-increasing effect of IspA was lower than the rescue effect, and it is considered that this is because IspA is 2-step enzyme, and DMAPP is converted into FPP without releasing intermediate GPP which is a direct substrate of GES_{M53}.

The overexpression of all the MEP enzymes, except for DXS, did not significantly show the rescue effect from GES_{M53} expression, and the dye production increasing effect was also slight. This result indicates none of the particular enzymes constitutes a clear bottleneck in the MEP pathway. That is, if the enzyme activity can be further improved by the evolution of not only the MEP enzymes but also the enzyme involved in the isoprenoid pathway, the dye production increasing effect can be obtained.

That is, screening of the enzyme involved in the isoprenoid pathway using the rescue effect from the GES expression toxicity as an index is enabled.

### Example 3

### (Screening of Activity of Enzyme Involved in Isoprenoid Pathway)

From the results of b of FIG. 15, it is unlikely that any one of the enzymes in the MEP pathway causes a clear rate-limiting step. That is, it is considered that only an activity of one MEP enzyme increases and the titer of the MEP pathway does not greatly change. The reason why DXS had a significant effect is that this enzyme is in the first step of the pathway rather than in the rate-limiting step and is considered to compete with an intrinsic metabolic pathway.

It was determined that IspH (c of FIG. 15) for simultaneously supplying two products (DMAPP and IPP) in the final step of the MEP pathway is the good target for improving the titer of the MEP pathway than other enzymes. IspH has clear product selectivity, and the intracellular supply amounts of DMAPP and IPP are fixed to 1:5.

Thus, IspH was selected as the enzyme involved in the isoprenoid pathway. Details are as follows.

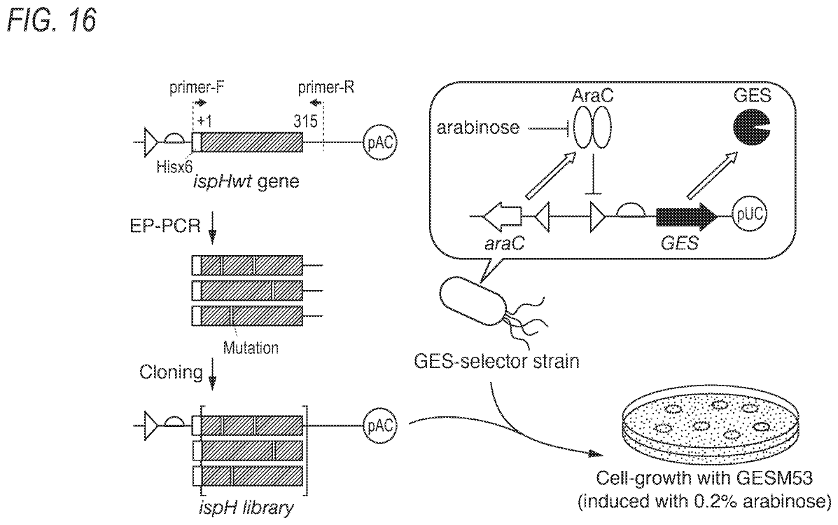

Random mutations were introduced into the whole gene of the IspH gene to prepare an IspH library (diversity: 4,650) (FIG. 16). The IspH library was introduced into the Escherichia coli strain BW25113(Δtna) in which GES_{M53} was expressed, and the Escherichia coli strain BW25113(Δtna) was spread on an arabinose-containing agar medium.

On the arabinose medium on which 5,000 cells had been spread, 15 colonies were generated. Among these transformants, there were four transformants that could not form colonies on the arabinose medium in a retransformation experiment. it was confirmed that the remaining 11 transformants could rescue a host from GES_{M53} expression with good reproducibility.

The genotypes of the 15 transformants are shown in the following Table 1. It was confirmed that the genotypes of 11 transformants had four genotypes that are “Thr207Ser, Glu301Asp”, “Thr175Ser”, “Leu105Gln”, and “Gln211Leu, Gln220Arg, Asn306Asp”.

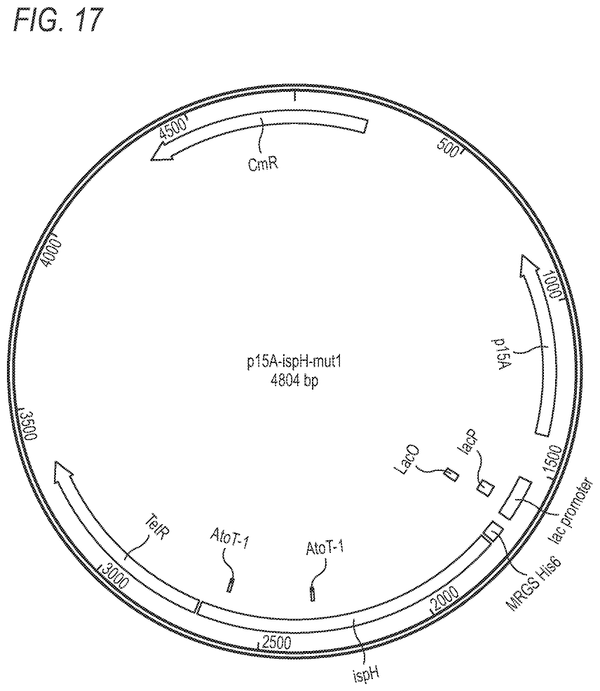

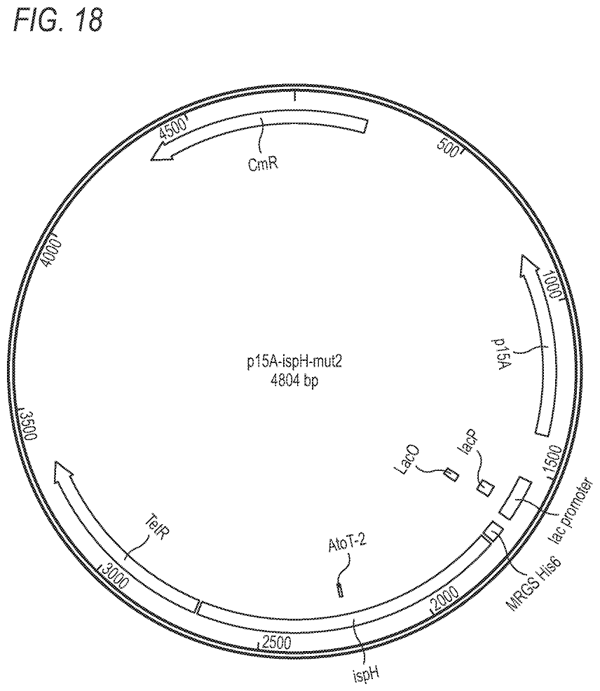

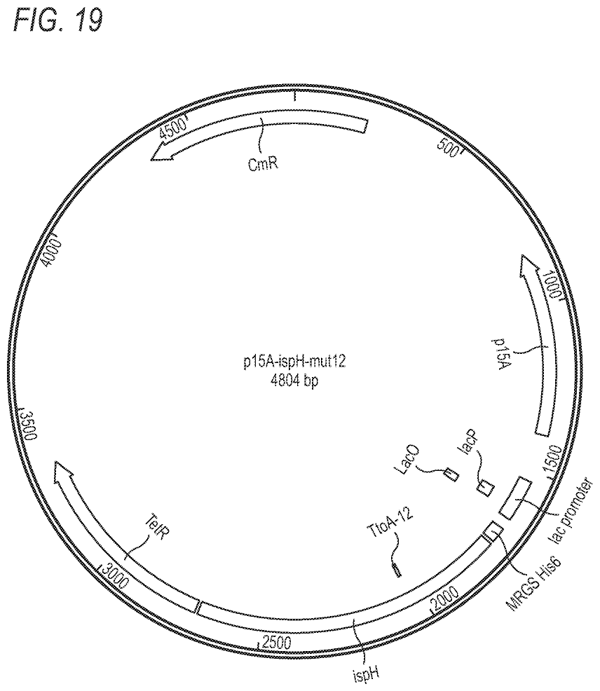

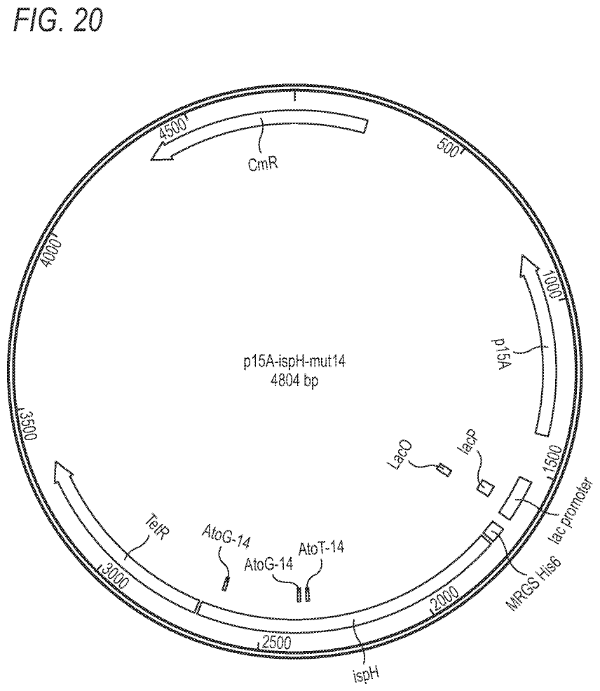

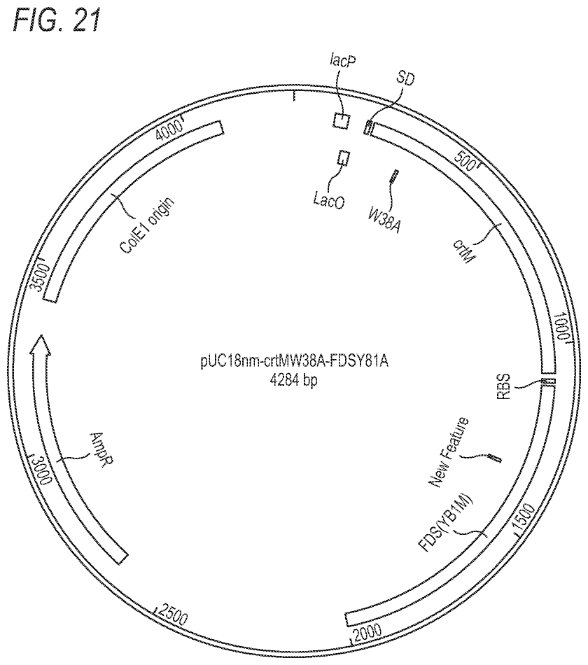

FIGS. 17 to 21 show the used plasmids p15A-ispHₘᵤₜ₁, p15A-ispHₘᵤₜ₂, p15A-ispHₘᵤₜ₁₂, p15A-ispHₘᵤₜ₁₄, and pUC18 nm-Crt_{MW38A}-FDS_{Y81A}.

TABLE 1

| Genotypes of | |
|---|---|
| IspH mutants | |
| IspH_{Mut1} | Thr207Ser, Glu301Asp |
| IspH_{Mut2} | Thr175Ser |
| IspH_{Mut3} | Thr207Ser, Glu301Asp |
| IspH_{Mut4} | (Determined as false-P in 2^{nd} screening) |
| IspH_{Mut5} | (Determined as false-P in 2^{nd} screening) |
| IspH_{Mut6} | Thr207Ser, Glu301Asp |
| IspH_{Mut7} | (Determined as false-P in 2^{nd} screening) |
| IspH_{Mut8} | Thr207Ser, Glu301Asp |
| IspH_{Mut9} | Thr207Ser, Glu301Asp |
| IspH_{Mut10} | Thr175Ser |
| IspH_{Mut11} | (Determined as false-P in 2^{nd} screening) |
| IspH_{Mut12} | Leu105Gln |
| IspH_{Mut13} | Leu105Gln |
| IspH_{Mut14} | Gln211Leu, Gln220Arg, Asn306Asp |
| IspH_{Mut15} | Thr207Ser, Glu301Asp |

### Example 4

### (Measurement of Enzyme Activity of IspH Mutant)

In the present Example, it was checked whether the IspH mutants having four genotypes obtained in Example 3 actually are with improved precursor supplying capacity (a dye production increasing effect). Details are as follows.

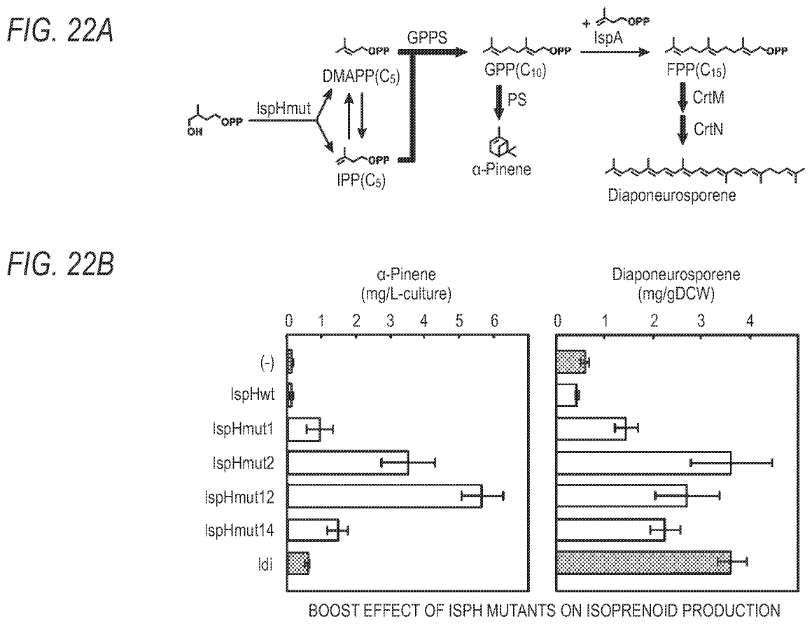

As shown in a of FIG. 22, in order to enable expression of α-pinene and diaponeurosporene, expression plasmids for mutants having four genotypes were introduced together with expression vectors of pinene synthase (PtPS) and a diaponeurosporene synthesis pathway (CrtM, CrtN) into the Escherichia coli strain (BW25113), and the production amounts of α-pinene and diaponeurosporene were checked (b of FIG. 22).

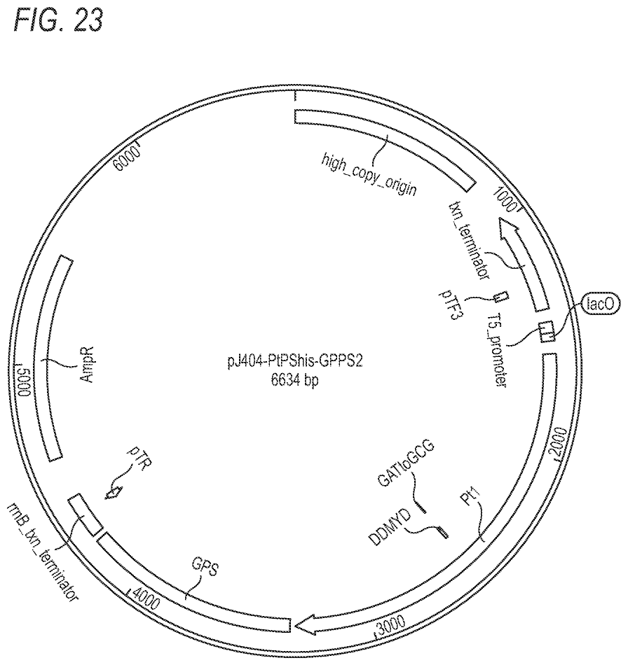

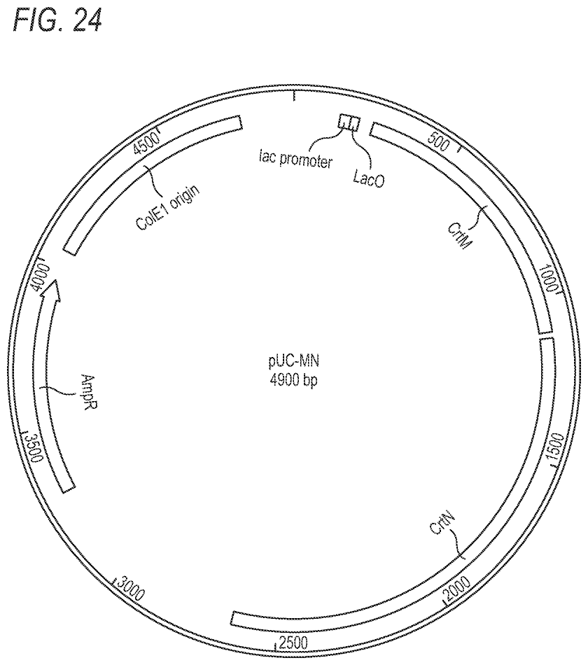

Specifically, BW25113Δtna strains into which a pinene synthesis plasmid (pJ404-PShis-GPPS) or a diaponeurosporene synthesis plasmid (pUC-CrtM-CrtN) and an expression plasmid of a wild-type IspH or IspH mutants (p15A-ispHwt, p15A-ispHₘᵤₜ₁, p15A-ispHₘᵤₜ₂, p15A-ispHₘᵤₜ₁₂, p15A-ispHₘᵤₜ₁₄) were co-introduced were subjected to shaking culture for 48 hours, and the production amounts thereof were analyzed by a previously reported method (GC-FID was used for pinene, and HPLC-PDA-MS was used for diaponeurosporene). As a control, the same experiment was performed using a plasmid expressing idi instead of the IspH mutant. FIGS. 23 to 24 show the expression plasmids of the diaponeurosporene enzyme (pJ404-PShis-GPPS) and the pinene enzyme (pUC-crtM-crtN).

All the gene mutant types exhibited the effect of increasing the production of α-pinene and diaponeurosporene (b of FIG. 22). Regarding the synthesis of diaponeurosporene using FPP as a raw material, the wild-type IspH did not exhibit a production-increasing effect. On the other hand, all IspH mutants exhibited a production-increasing effect of about 3 times to 8 times in single enhancement expression. Regarding the synthesis of a pinene which was a monoterpene using GPP as a raw material, a production-increasing effect of several tens of times by single enhancement of IspHₘᵤₜ₁₂ was exhibited.

### Example 5

### (Improvement in Enzyme Activity of IspH Mutant)

In general, it is difficult to assume that the activity (Vmax) of an enzyme becomes several tens of times by only the substitution of one amino acid (Leu105Gln). Further, the effect of increasing the production of isoprenoids reflects the flux of the entire MEP pathway, and it is clear that the IspH process is not a rate-limiting step in the MEP pathway based on the fact that the overexpression of the IspH wild type does not show the production-increasing effect at all (a of FIG. 15 and b of FIG. 22).

IspHₘᵤₜ₁₂ showing the highest performance in pinene production is inferior to IspHₘᵤₜ₂ in an effect of increasing the production of diaponeurosporene using FPP as a raw material. The former is made from one molecule of DMAPP and one molecule of IPP (DMAPP:IPP=1:1), and the latter is made from two molecules of DMAPP and four molecules of IPP (DMAPP:IPP=1:2). If IspHₘᵤₜ₁₂ temporarily contributes not only to Vmax but also to the increase of the DMAPP ratio by changing the product selectivity, it matches the pinene production increasing effect shown in the results of b of FIG. 22.

Accordingly, it was clarified how IspHₘᵤₜ₁₂, which is an IspH mutant, increased the production amount of monoterpenes. Details are as follows.

In order to examine the product specificity (a ratio of DMAPP:IPP) of the IspH mutant, an expression vector for expressing an FDS mutant (FDSY81A) and a CrtM mutant (CrtMW38A) having low enzyme function specificity was used. Both mutants are reduced in the specificity by the amino acid substitution in a reaction pocket, and intentionally become enzyme mutants having low specificity. The FDS mutant can release C15, C20, or C25PP as a product, and the CrtM mutant can synthesize C30, C35, C40, or C45 carotenoid using the FDS mutant as a raw material.

Specifically, the IspH mutants were introduced into a cell BW25113Δtna strain into which pUC18 nm-CrtMW38A-FDSY81A had been introduced. An expression plasmid for the mutant was additionally introduced, followed by shaking culture at 30° C. for 48 hours. The culture solution was collected, extracted with acetone, and extracted with hexane or physiological saline to obtain and concentrate a hexane fraction. The concentrated hexane fraction was dissolved in THF:acetonitrile=6:4 to obtain a sample. The sample was analyzed by HPLC-PDA-MS (mobile phase:acetonitrile:THF:methanol=58:4:38). Here, the FDS mutant (FDSY81A) and the CrtM mutant (CrtMW38A) were co-expressed. Both mutants are subjected to amino acid substitutions in the reaction pocket to relax the specificity thereof and intentionally become the enzyme mutant with low specificity. The FDS mutant can release C15, C20, or C25PP as a product, and the CrtM mutant can synthesize C30, C35, C40, or C45 carotenoid using the FDS mutant as a raw material. That is, a system with extremely low selectivity was constructed as a pathway (see: Furubayashi et al., Nat. Commun., 6, 7534 (2015)). If the DMAPP/IPP ratio in cells increases temporarily, a carotenoid having a lower molecular weight is synthesized in the carotenoid pathway having low specificity.

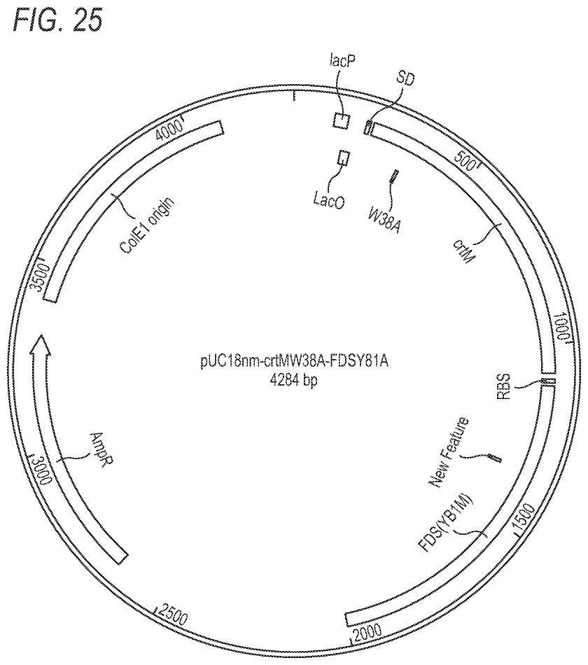

FIG. 25 shows an expression plasmid of pUC18 nm-CrtM_{W38A}-FDS_{Y81A}.

FIG. 26 shows the results of HPLC analysis of carotenoid fractions accumulated in the carotenoid pathway in which the FDS mutant and the CrtM mutant coexist with the IspH mutant. In the case where the FDS mutant and the CrtM mutant coexist with the wild-type IspH, C30 and C45 carotenoids are synthesized mainly around a C40 pathway. In the case where the FDS mutant and the CrtM mutant coexist with Idi, the production amount is greatly improved without changing the ratio of the carotenoid using the wild-type IspH (C40 carotenoid is a main product). The IspHₘᵤₜ₁ showed the same results as the ratio of carotenoid using the wild-type IspH.

On the other hand, it was confirmed that the products using IspHₘᵤₜ₂, IspHₘᵤₜ₁₂, and IspHₘᵤₜ₁₄ were clearly biased to the low-molecular-weight-side as compared with the wild-type IspH. This is because the isoprenoid raw material to which the IspH mutant is supplied biases to DMAPP (DMAPP:IPP ratio is high).

The carotenoid and the terpene use DMAPP as a starting raw material, and a plurality of IPPs are added thereto to provide a direct substrate of each terpene (in the case of the pinene material GPP which is monoterpene, one IPP is consumed per DMAPP, in the case of FPP which is a raw material of farnesene in sesquiterpenes, or squalene that is a triterpene, or diaponeurosporene, two IPPs are consumed per DMAPP, in the case of GGPP which is a raw material of diterpenes or tetraterpenes, three IPPs are consumed per DMAPP, in the case of the quinone side chain C40 of Escherichia coli, seven IPPs are consumed per DMAPP, and in the case of the Escherichia coli bactoprenol, 10 IPPs are consumed per DMAOH).

That is, it is considered that the size distribution of carotenoid skeletons provided by the carotenoid biosynthesis pathway with low size-selectivity reflects the presence amount of IPP and DMAPP as precursors (DMAPP:IPP ratio), and thus results as shown in FIG. 26 are obtained.

It was clarified that it is important to increase the supply amount of DMAPP (increase DMAPP:IPP ratio) in the case of low-molecular-weight (low-boiling-point) terpenes, monoterpenes, and hemiterpenes (C5, isoprene gas) for the production of valuable terpenoids. In the Escherichia coli host, the main use of the isoprenoid pathway is quinone side chain molecules (C40, DMAPP:IPP=1:7) and bactoprenol (C55, DMAOH:IPP=1:10). In response to the demand for a metabolism network of a host, the supply ratio of DMAPP:IPP in the MEP pathway is set as 1:5 by default (see: Altincicek et al., FEBS Lett., 532, 437-440 (2002)).

According to the present examples, it was confirmed that in order to efficiently produce low-molecular-weight terpenes (products having a high degree of demand for DMAPP), it is important not only to increase the metabolic flux but also to increase the DMAPP/IPP ratio.

That is, when IspHₘᵤₜ₁ is used, C45 and C40 can be efficiently synthesized by decreasing the DMAPP/IPP ratio. In addition, when IspH_{mut2,12,14} are used, C35 and C30 can be efficiently synthesized by increasing the DMAPP/IPP ratio.

### Example 6

### (Isoprenoid Synthesis Via Non-Mevalonate Pathway Using IspH Mutant)

An isoprenoid synthesis system was constructed via a non-mevalonate pathway in which IspHₘᵤₜ₁₂, which is an IspH mutant created in the above Example, and further, Dxs, IspG, and Dxr, which are enzymes involved in the non-mevalonate pathway, expressed in an enhanced manner, and the production level of α-pinene, which is an isoprenoid, was confirmed. Details are as follows.

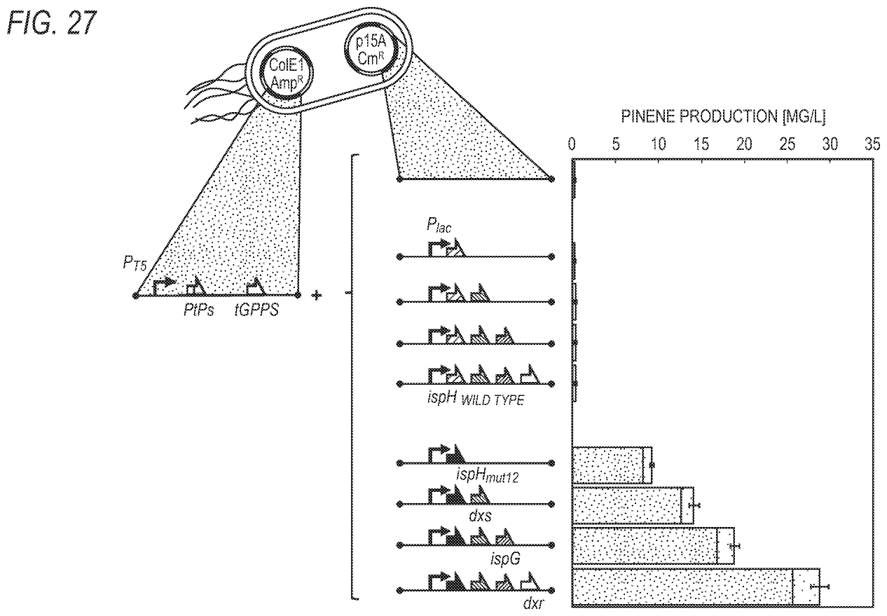

Operons capable of expressing Dxs, IspG, and/or Dxr respectively in IspH or IspHₘᵤₜ₁₂ and a plasmid for co-expressing GPPS and PtPS were introduced into an Escherichia coli strain (see: FIG. 27). The transformant was continuously extracted to dodecane in the uppermost layer while being cultured in a TB medium. The dodecane was collected from each batch, and a pinene extracted therefrom was quantified and compared according to a gas chromatography-FID detection method.

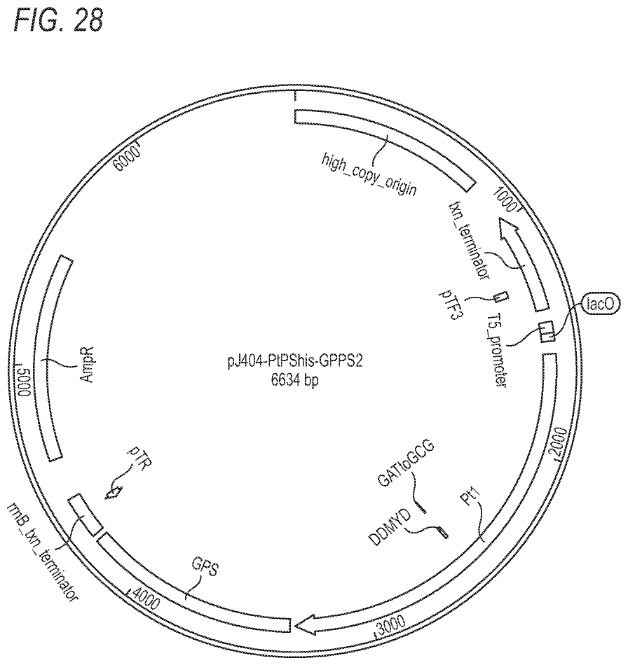

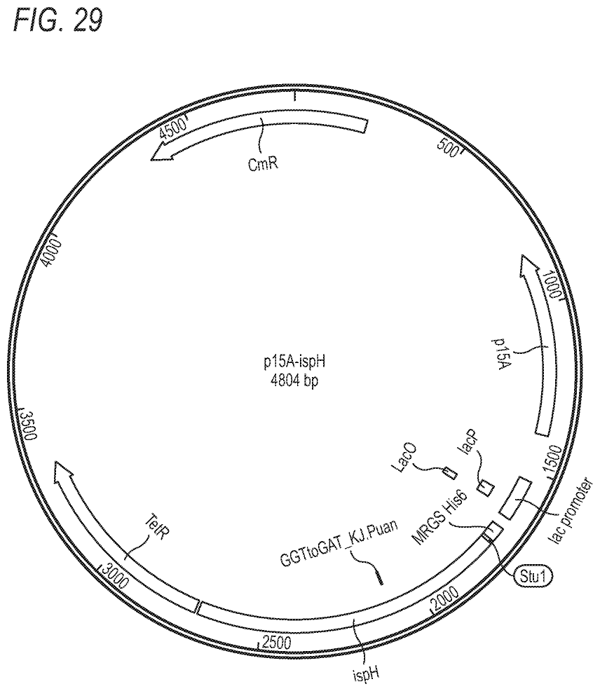

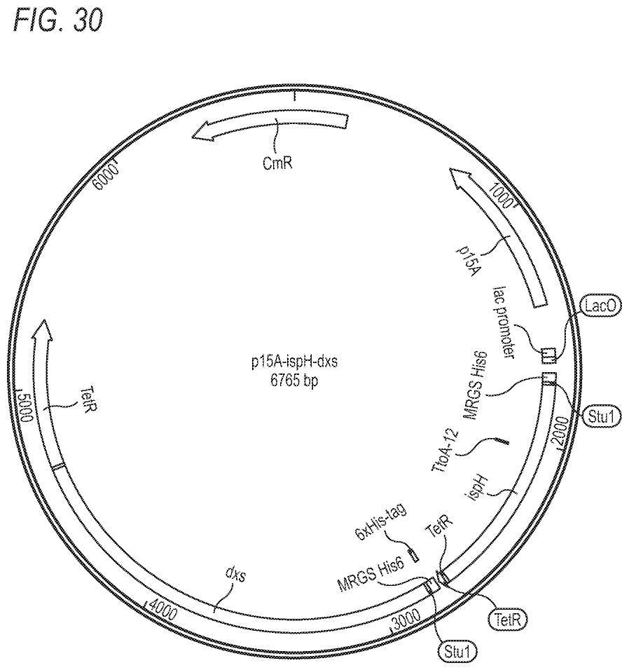

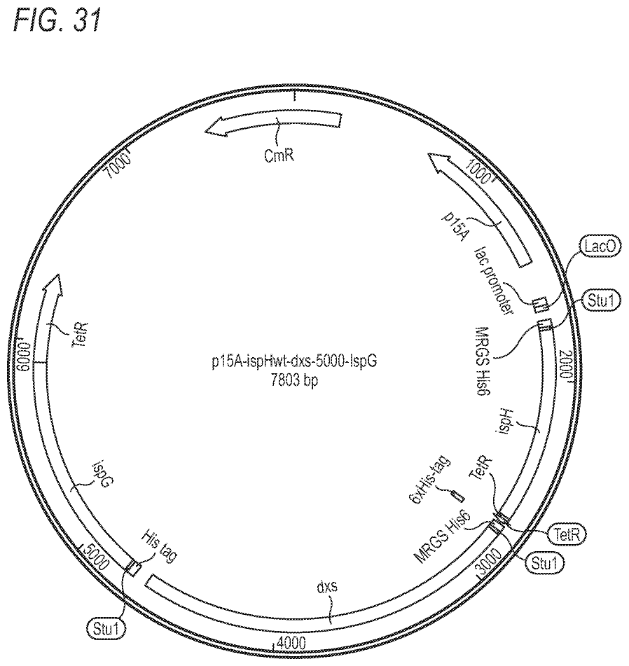

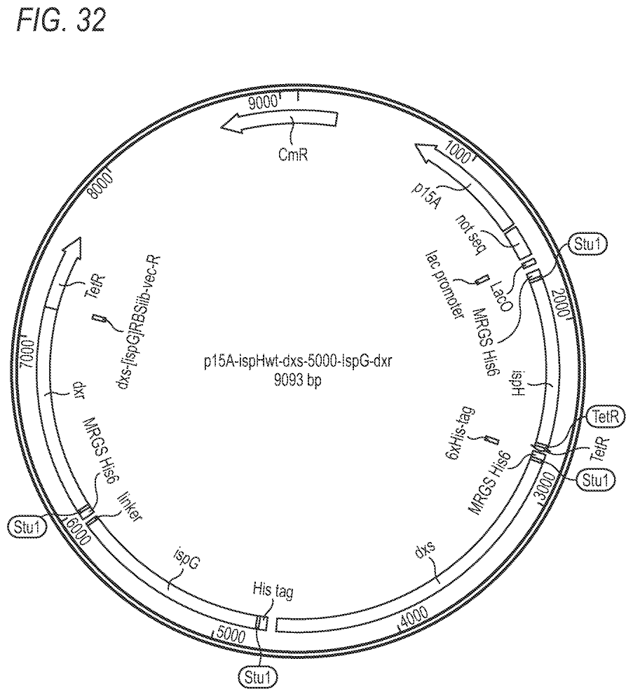

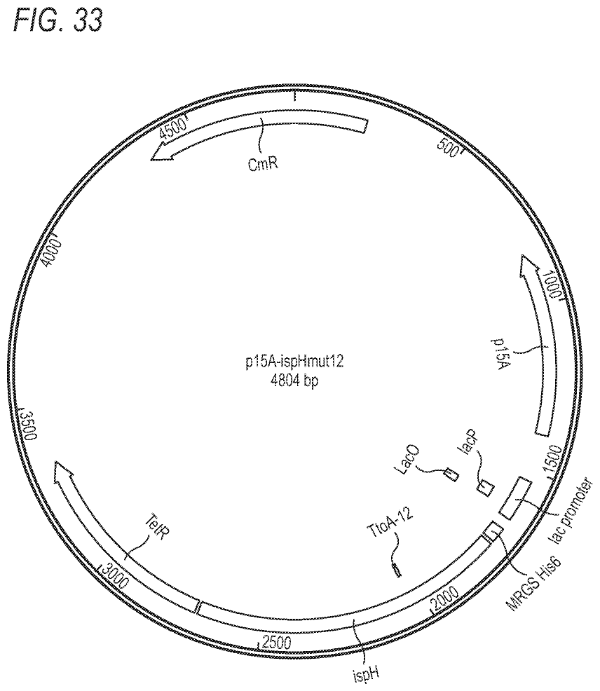

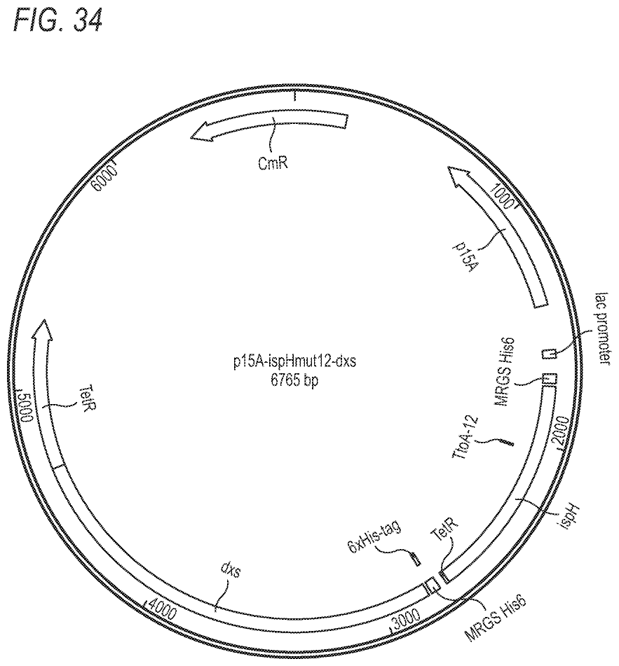

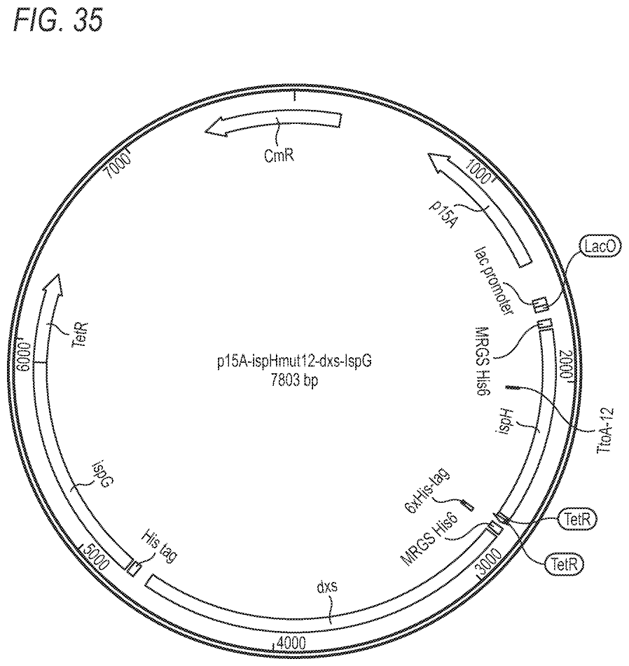

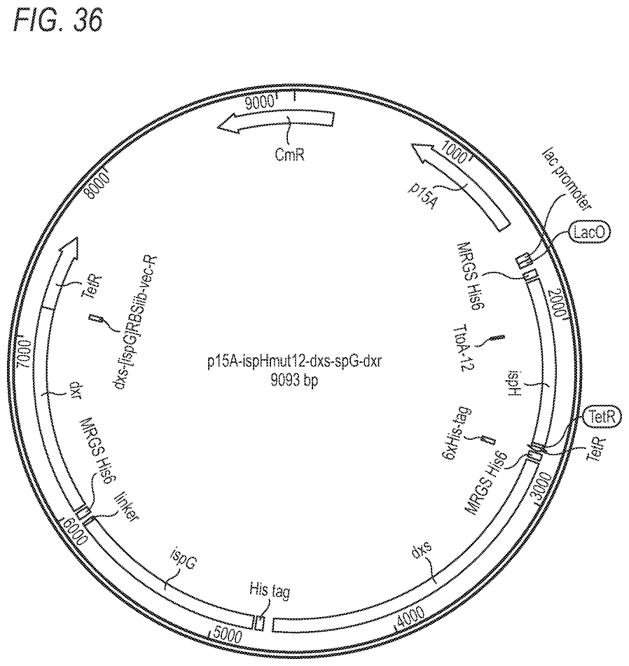

FIGS. 28 to 36 showed maps of the used plasmids (pJ404-PtPS_{Q457L}-GPPS, p15A-ispH_{wt}, p15A-ispH_{wt}-dxs, p15A-ispH_{wt}-dxs-ispG, p15A-ispH_{wt}-dxs-ispG-dxr, p15A-P_{lac}/lacO-ispHₘᵤₜ₁₂, p15A-ispHₘᵤₜ₁₂-dxs, p15A-ispHₘᵤₜ₁₂-dxs-ispG, and p15A-ispHₘᵤₜ₁₂-dxs-ispG-dxr).

Regarding the synthesis system using the IspH wild type, the production amount was flat (FIG. 27) even when another biosynthetic genetic gene was additionally expressed. On the other hand, regarding IspHₘᵤₜ₁₂, not only the production-increasing effect of 30 times or more was exhibited even by itself, but also a net increase in the production amount was obtained and a production amount exceeding about 100 times was achieved every time dxs, ispG, and dxr were added (FIG. 27).

### (Outline)

In the prior research, as shown in FIG. 37, the MEV pathway has been successfully improved, but the MEP pathway has not been successfully improved. It is considered that the MEP pathway exceeding MEV in terms of a carbon yield has multiple controls that have not been clarified, and an increase in production is limited. In the related art, the initial reaction (dxs) has been considered to be a rate-limiting factor in an enzyme step of 7-steps of the MEP pathway. However, it was clarified in the present Example that IspH in the final reaction was a rate-limiting factor. In the present example, the production amount of terpenes in Escherichia coli containing the MEP pathway into which the IspH mutant was introduced could be increased by 170 times.

In addition, in order to simultaneously develop both pathways that are the MEV pathway and the MEP pathway with high efficiency, it is necessary to increase the DMAPP/IPP ratio depending on the pathways of FIG. 37. As is clear from the results of Example 5 in the present invention, when IspHmut_{2,12,14} in the present invention are used, both pathways that are the MEV pathway and the MEP pathway can be simultaneously caused to express with high efficiency by increasing the DMAPP/IPP ratio.

### INDUSTRIAL APPLICABILITY

The present invention can provide a mutant IspH by which a production level of an isoprenoid is higher than that of a wild-type IspH, and an isoprenoid synthesis method using the mutant IspH.

1. A mutant-IspH gene comprising any one gene of the following (1) to (8):
(1) a gene encoding a polypeptide consisting of the amino acid sequence shown by SEQ ID NO: 5, 7, 9, or 11;
(2) a gene encoding a polypeptide in which 1 to 30 amino acids are substituted, inserted, deleted, and/or added in the amino acid sequence shown by SEQ ID NO: 5, 7, 9, or 11, and the polypeptide has an activity substantially equivalent to the activity of the polypeptide consisting of the amino acid sequence shown by SEQ ID NO: 5, 7, 9, or 11 of generating IPP (isopentenyl diphosphate) and/or DMAPP (dimethylallyl diphosphate) by using HMB-PP {(E)-4-hydroxy-3-methyl-2-butenyl diphosphate} as a substrate;
(3) a gene encoding a polypeptide which has an identity of 80% or more with the amino acid sequence shown by SEQ ID NO: 5, 7, 9, or 11, and which has an activity substantially equivalent to an activity of the polypeptide consisting of the amino acid sequence shown by SEQ ID NO: 5, 7, 9, or 11 of generating IPP and/or DMAPP by using HMB-PP as a substrate;
(4) a gene consisting of a DNA consisting of the nucleotide sequence shown by SEQ ID NO: 6, 8, 10, or 12;
(5) a gene which hybridizes with a DNA consisting of a nucleotide sequence complementary to the nucleotide sequence shown by SEQ ID NO: 6, 8, 10, or 12 under a stringent condition, and which encodes a polypeptide having an activity substantially equivalent to the activity of the polypeptide consisting of the amino acid sequence shown by SEQ ID NO: 5, 7, 9, or 11 of generating IPP and/or DMAPP by using HMB-PP as a substrate;
(6) a gene consisting of a DNA in which 1 to 50 nucleotides are substituted, inserted, deleted, and/or added in the DNA consisting of the nucleotide sequence shown by SEQ ID NO: 6, 8, 10, or 12;
(7) a gene consisting of a DNA having an identity of 80% or more with the DNA consisting of the nucleotide sequence shown by SEQ ID NO: 6, 8, 10, or 12; and
(8) a gene consisting of a DNA consisting of a condensation isomer of the nucleotide sequence shown by SEQ ID NO: 6, 8, 10, or 12.

2. The mutant-IspH gene according to claim 1, wherein the gene is a gene encoding a polypeptide consisting of the amino acid sequence shown by SEQ ID NO: 5 or a gene consisting of a DNA consisting of the nucleotide sequence shown by SEQ ID NO: 6.

3. The mutant-IspH gene according to claim 1, wherein the gene is a gene encoding a polypeptide consisting of the amino acid sequence shown by SEQ ID NO: 7 or a gene consisting of a DNA consisting of the nucleotide sequence shown by SEQ ID NO: 8.

4. The mutant-IspH gene according to claim 1, wherein the gene is a gene encoding a polypeptide consisting of the amino acid sequence shown by SEQ ID NO: 9 or a gene consisting of a DNA consisting of the nucleotide sequence shown by SEQ ID NO: 10.

5. The mutant-IspH gene according to claim 1, wherein the gene is a gene encoding a polypeptide consisting of the amino acid sequence shown by SEQ ID NO: 11 or a gene consisting of a DNA consisting of the nucleotide sequence shown by SEQ ID NO: 12.

6. A method for synthesizing a compound having, as a structural unit, isoprene, the method comprising: using a cell into which the mutant IspH gene according to claim 1 is introduced, wherein the cell has a higher activity for synthesizing isopentenyl diphosphate and/or dimethylallyl diphosphate than a wild-type cell.

7. (canceled)

8. A mutant IspH comprising any one peptide of the following (1) to (7):
(1) a polypeptide consisting of the amino acid sequence shown by SEQ ID NO: 5, 7, 9, or 11;
(2) a polypeptide which consists of an amino acid sequence in which 1 to 30 amino acids are substituted, deleted, inserted, and/or added in the amino acid sequence shown by SEQ ID NO: 5, 7, 9, or 11, and which has an activity substantially equivalent to an activity of the polypeptide consisting of the amino acid sequence shown by SEQ ID NO: 5, 7, 9, or 11 of generating IPP and/or DMAPP by using HMB-PP as a substrate;
(3) a polypeptide which consists of an amino acid sequence having an identity of 80% or more with the amino acid sequence shown by SEQ ID NO: 5, 7, 9, or 11, and which has an activity substantially equivalent to the activity of the polypeptide consisting of the amino acid sequence shown by SEQ ID NO: 5, 7, 9, or 11 of generating IPP and/or DMAPP by using HMB-PP as a substrate;
(4) a peptide consisting of an amino acid sequence encoded by the nucleotide sequence shown by SEQ ID NO: 6, 8, 10, or 12;
(5) a peptide which consists of an amino acid sequence encoded by a DNA which hybridizes with a DNA consisting of a nucleotide sequence complementary to the nucleotide sequence shown by SEQ ID NO: 6, 8, 10, or 12 under a stringent condition, and which has an activity substantially equivalent to the activity of the polypeptide consisting of the amino acid sequence shown by SEQ ID NO: 5, 7, 9, or 11 of generating IPP and/or DMAPP by using HMB-PP as a substrate;
(6) a peptide consisting of an amino acid sequence encoded by a DNA in which 1 to 50 nucleotides are substituted, deleted, inserted, and/or added in the DNA consisting of the nucleotide sequence shown by SEQ ID NO: 6, 8, 10, or 12; and
(7) a peptide consisting of an amino acid sequence encoded by a DNA having an identity of 80% or more with the DNA consisting of the nucleotide sequence shown by SEQ ID NO: 6, 8, 10, or 12.

9. A mutant IspH or a derivative thereof comprising a polypeptide consisting of an amino acid sequence which has amino acid sequence represented by SEQ ID NO: 1 and has an amino acid substitution at 1, 2, 3, 4, 5, 6, or 7 sites selected from the group consisting of the 105th position, the 175th position, the 207th position, the 211th position, the 220th position, the 301st position, and the 306th position.

10. The mutant IspH or the derivative thereof according to claim 9, wherein the mutant IspH has a higher activity for synthesizing isopentenyl diphosphate and/or dimethylallyl diphosphate than wild-type IspH.

11. The mutant IspH or the derivative thereof according to claim 9, wherein the amino acid substitution is at least one amino acid substitution selected from the following group:
Leu105Gln, Thr175Ser, Thr207Ser, Gln211Leu, Gln220Arg, Glu301Asp, and Asn306Asp.

12. The mutant IspH or the derivative thereof according to claim 9, wherein the amino acid substitution is selected from any one or more of the following (1) to (4):
(1) Thr207Ser and/or Glu301Asp;
(2) Thr175Ser;
(3) Leu105Gln; and
(4) Gln211Leu, Gln220Arg, and/or Asn306Asp.

13. The mutant IspH or the derivative thereof according to claim 12, wherein the amino acid substitution is Thr207Ser and/or Glu301Asp.

14. The mutant IspH or the derivative thereof according to claim 12, wherein the amino acid substitution is Thr175Ser.

15. The mutant IspH or the derivative thereof according to claim 12, wherein the amino acid substitution is Leu105Gln.

16. The mutant IspH or the derivative thereof according to claim 12, wherein the amino acid substitution is are Gln211Leu, Gln220Arg and/or Asn306Asp.

17. A method for screening a mutant enzyme involved in an isoprenoid pathway, the method comprising:
introducing a gene library of an enzyme involved in an isoprenoid pathway into a cell with an enhanced enzyme activity for geraniol, a mutation being introduced into the gene library; and
using an increase, a decrease, or a loss in a cell growth rate of the cell as an index.

18. The screening method according to claim 17, wherein the increase in the cell growth rate of the cell is an increase in the production level of α-pinene and/or diaponeurosporene.

19. The screening method according to claim 17, wherein the cell is Escherichia coli.

20. The mutant IspH or the derivative thereof according to claim 10, wherein the amino acid substitution is at least one amino acid substitution selected from the following group:
Leu105Gln, Thr175Ser, Thr207Ser, Gln211Leu, Gln220Arg, Glu301Asp, and Asn306Asp.

21. The screening method according to claim 18, wherein the cell is Escherichia coli.


SEQUENCE LISTING (39 sequences)

SEQ ID NO: 1 - protein, 323 aa, Escherichia coli
MTDPALRAQILLANPRGFCAGVDRAISIVENALAIYGAPIYVRHEVVHNRYVVDSLRERGAIFIEQISEVPDGAILIFSAHGVSQAVRNEAKSRDLTVFDATCPLVTKVHMEVARASRRGEESILIGHAGHPEVEGTMGQYSNPEGGMYLVESPDDVWKLTVKNEEKLSFMTQTTLSVDDTSDVIDALRKRFPKIVGPRKDDICYATTNRQEAVRALAEQAEVVLVVGSKNSSNSNRLAELAQRMGKRAFLIDDAKDIQEEWVKEVKCVGVTAGASAPDILVQNVVARLQQLGGGEAIPLEGREENIVFEVPKELRVDIREVD

SEQ ID NO: 2 - DNA, 972 bp, Escherichia coli
atgacggatccggccctgagggcccagatcctgttggccaacccgcgtggtttttgtgccggggtagaccgcgctatcagcattgttgaaaacgcgctggccatttacggcgcaccgatatatgtccgtcacgaagtggtacataaccgctatgtggtcgatagcttgcgtgagcgtggggctatctttattgagcagattagcgaagtaccggacggcgcgatcctgattttctccgcacacggtgtttctcaggcggtacgtaacgaagcaaaaagtcgcgatttgacggtgtttgatgccacctgtccgctggtgaccaaagtgcatatggaagtcgcccgcgccagtcgccgtggcgaagaatctattctcatcggtcacgccgggcacccggaagtggaagggacaatgggccagtacagtaacccggaagggggaatgtatctggtcgaatcgccggacgatgtgtggaaactgacggtcaaaaacgaagagaagctctcctttatgacccagaccacgctgtcggtggatgacacgtctgatgtgatcgacgcgctgcgtaaacgcttcccgaaaattgtcggtccgcgcaaagatgacatctgctacgccacgactaaccgtcaggaagcggtacgcgccctggcagaacaggcggaagttgtgttggtggtcggttcgaaaaactcctccaactccaaccgtctggcggagctggcccagcgtatgggcaaacgcgcgtttttgattgacgatgcgaaagacatccaggaagagtgggtgaaagaggttaaatgcgtcggcgtgactgcgggcgcatcggctccggatattctggtgcagaatgtggtggcacgtttgcagcagctgggcggtggtgaagccattccgctggaaggccgtgaagaaaacattgttttcgaagtgccgaaagagctgcgtgtcgatattcgtgaagtcgattaa

SEQ ID NO: 3 - protein, 332 aa, Artificial Sequence
  Wild-type IspH amino acid sequence with “RGSHHHHHH” insertion, which used in Example
MRGSHHHHHHTDPALRAQILLANPRGFCAGVDRAISIVENALAIYGAPIYVRHEVVHNRYVVDSLRERGAIFIEQISEVPDGAILIFSAHGVSQAVRNEAKSRDLTVFDATCPLVTKVHMEVARASRRGEESILIGHAGHPEVEGTMGQYSNPEGGMYLVESPDDVWKLTVKNEEKLSFMTQTTLSVDDTSDVIDALRKRFPKIVGPRKDDICYATTNRQEAVRALAEQAEVVLVVGSKNSSNSNRLAELAQRMGKRAFLIDDAKDIQEEWVKEVKCVGVTAGASAPDILVQNVVARLQQLGGGEAIPLEGREENIVFEVPKELRVDIREVD

SEQ ID NO: 4 - DNA, 999 bp, Artificial Sequence
  Wild-type IspH nucleotide sequence with “agaggatctcaccatcaccatcaccat” insertion, which used in Example
atgagaggatctcaccatcaccatcaccatacggatccggccctgagggcccagatcctgttggccaacccgcgtggtttttgtgccggggtagaccgcgctatcagcattgttgaaaacgcgctggccatttacggcgcaccgatatatgtccgtcacgaagtggtacataaccgctatgtggtcgatagcttgcgtgagcgtggggctatctttattgagcagattagcgaagtaccggacggcgcgatcctgattttctccgcacacggtgtttctcaggcggtacgtaacgaagcaaaaagtcgcgatttgacggtgtttgatgccacctgtccgctggtgaccaaagtgcatatggaagtcgcccgcgccagtcgccgtggcgaagaatctattctcatcggtcacgccgggcacccggaagtggaagggacaatgggccagtacagtaacccggaagggggaatgtatctggtcgaatcgccggacgatgtgtggaaactgacggtcaaaaacgaagagaagctctcctttatgacccagaccacgctgtcggtggatgacacgtctgatgtgatcgacgcgctgcgtaaacgcttcccgaaaattgtcggtccgcgcaaagatgacatctgctacgccacgactaaccgtcaggaagcggtacgcgccctggcagaacaggcggaagttgtgttggtggtcggttcgaaaaactcctccaactccaaccgtctggcggagctggcccagcgtatgggcaaacgcgcgtttttgattgacgatgcgaaagacatccaggaagagtgggtgaaagaggttaaatgcgtcggcgtgactgcgggcgcatcggctccggatattctggtgcagaatgtggtggcacgtttgcagcagctgggcggtggtgaagccattccgctggaaggccgtgaagaaaacattgttttcgaagtgccgaaagagctgcgtgtcgatattcgtgaagtcgattaa

SEQ ID NO: 5 - protein, 323 aa, Artificial Sequence
  IspHmut1
MTDPALRAQILLANPRGFCAGVDRAISIVENALAIYGAPIYVRHEVVHNRYVVDSLRERGAIFIEQISEVPDGAILIFSAHGVSQAVRNEAKSRDLTVFDATCPLVTKVHMEVARASRRGEESILIGHAGHPEVEGTMGQYSNPEGGMYLVESPDDVWKLTVKNEEKLSFMTQTTLSVDDTSDVIDALRKRFPKIVGPRKDDICYASTNRQEAVRALAEQAEVVLVVGSKNSSNSNRLAELAQRMGKRAFLIDDAKDIQEEWVKEVKCVGVTAGASAPDILVQNVVARLQQLGGGEAIPLDGREENIVFEVPKELRVDIREVD

SEQ ID NO: 6 - DNA, 969 bp, Artificial Sequence
  IspHmut1
atgacggatccggccctgagggcccagatcctgttggccaacccgcgtggtttttgtgccggggtagaccgcgctatcagcattgttgaaaacgcgctggccatttacggcgcaccgatatatgtccgtcacgaagtggtacataaccgctatgtggtcgatagcttgcgtgagcgtggggctatctttattgagcagattagcgaagtaccggacggcgcgatcctgattttctccgcacacggtgtttctcaggcggtacgtaacgaagcaaaaagtcgcgatttgacggtgtttgatgccacctgtccgctggtgaccaaagtgcatatggaagtcgcccgcgccagtcgccgtggcgaagaatctattctcatcggtcacgccgggcacccggaagtggaagggacaatgggccagtacagtaacccggaagggggaatgtatctggtcgaatcgccggacgatgtgtggaaactgacggtcaaaaacgaagagaagctctcctttatgacccagaccacgctgtcggtggatgacacgtctgatgtgatcgacgcgctgcgtaaacgcttcccgaaaattgtcggtccgcgcaaagatgacatctgctacgcctcgactaaccgtcaggaagcggtacgcgccctggcagaacaggcggaagttgtgttggtggtcggttcgaaaaactcctccaactccaaccgtctggcggagctggcccagcgtatgggcaaacgcgcgtttttgattgacgatgcgaaagacatccaggaagagtgggtgaaagaggttaaatgcgtcggcgtgactgcgggcgcatcggctccggatattctggtgcagaatgtggtggcacgtttgcagcagctgggcggtggtgaagccattccgctggatggccgtgaagaaaacattgttttcgaagtgccgaaagagctgcgtgtcgatattcgtgaagtcgat

SEQ ID NO: 7 - protein, 323 aa, Artificial Sequence
  IspHmut2
MTDPALRAQILLANPRGFCAGVDRAISIVENALAIYGAPIYVRHEVVHNRYVVDSLRERGAIFIEQISEVPDGAILIFSAHGVSQAVRNEAKSRDLTVFDATCPLVTKVHMEVARASRRGEESILIGHAGHPEVEGTMGQYSNPEGGMYLVESPDDVWKLTVKNEEKLSFMTQTSLSVDDTSDVIDALRKRFPKIVGPRKDDICYATTNRQEAVRALAEQAEVVLVVGSKNSSNSNRLAELAQRMGKRAFLIDDAKDIQEEWVKEVKCVGVTAGASAPDILVQNVVARLQQLGGGEAIPLEGREENIVFEVPKELRVDIREVD

SEQ ID NO: 8 - DNA, 969 bp, Artificial Sequence
  IspHmut2
atgacggatccggccctgagggcccagatcctgttggccaacccgcgtggtttttgtgccggggtagaccgcgctatcagcattgttgaaaacgcgctggccatttacggcgcaccgatatatgtccgtcacgaagtggtacataaccgctatgtggtcgatagcttgcgtgagcgtggggctatctttattgagcagattagcgaagtaccggacggcgcgatcctgattttctccgcacacggtgtttctcaggcggtacgtaacgaagcaaaaagtcgcgatttgacggtgtttgatgccacctgtccgctggtgaccaaagtgcatatggaagtcgcccgcgccagtcgccgtggcgaagaatctattctcatcggtcacgccgggcacccggaagtggaagggacaatgggccagtacagtaacccggaagggggaatgtatctggtcgaatcgccggacgatgtgtggaaactgacggtcaaaaacgaagagaagctctcctttatgacccagacctcgctgtcggtggatgacacgtctgatgtgatcgacgcgctgcgtaaacgcttcccgaaaattgtcggtccgcgcaaagatgacatctgctacgccacgactaaccgtcaggaagcggtacgcgccctggcagaacaggcggaagttgtgttggtggtcggttcgaaaaactcctccaactccaaccgtctggcggagctggcccagcgtatgggcaaacgcgcgtttttgattgacgatgcgaaagacatccaggaagagtgggtgaaagaggttaaatgcgtcggcgtgactgcgggcgcatcggctccggatattctggtgcagaatgtggtggcacgtttgcagcagctgggcggtggtgaagccattccgctggaaggccgtgaagaaaacattgttttcgaagtgccgaaagagctgcgtgtcgatattcgtgaagtcgat

SEQ ID NO: 9 - protein, 323 aa, Artificial Sequence
  IspHmut12
MTDPALRAQILLANPRGFCAGVDRAISIVENALAIYGAPIYVRHEVVHNRYVVDSLRERGAIFIEQISEVPDGAILIFSAHGVSQAVRNEAKSRDLTVFDATCPQVTKVHMEVARASRRGEESILIGHAGHPEVEGTMGQYSNPEGGMYLVESPDDVWKLTVKNEEKLSFMTQTTLSVDDTSDVIDALRKRFPKIVGPRKDDICYATTNRQEAVRALAEQAEVVLVVGSKNSSNSNRLAELAQRMGKRAFLIDDAKDIQEEWVKEVKCVGVTAGASAPDILVQNVVARLQQLGGGEAIPLEGREENIVFEVPKELRVDIREVD

SEQ ID NO: 10 - DNA, 969 bp, Artificial Sequence
  IspHmut12
atgacggatccggccctgagggcccagatcctgttggccaacccgcgtggtttttgtgccggggtagaccgcgctatcagcattgttgaaaacgcgctggccatttacggcgcaccgatatatgtccgtcacgaagtggtacataaccgctatgtggtcgatagcttgcgtgagcgtggggctatctttattgagcagattagcgaagtaccggacggcgcgatcctgattttctccgcacacggtgtttctcaggcggtacgtaacgaagcaaaaagtcgcgatttgacggtgtttgatgccacctgtccgcaggtgaccaaagtgcatatggaagtcgcccgcgccagtcgccgtggcgaagaatctattctcatcggtcacgccgggcacccggaagtggaagggacaatgggccagtacagtaacccggaagggggaatgtatctggtcgaatcgccggacgatgtgtggaaactgacggtcaaaaacgaagagaagctctcctttatgacccagaccacgctgtcggtggatgacacgtctgatgtgatcgacgcgctgcgtaaacgcttcccgaaaattgtcggtccgcgcaaagatgacatctgctacgccacgactaaccgtcaggaagcggtacgcgccctggcagaacaggcggaagttgtgttggtggtcggttcgaaaaactcctccaactccaaccgtctggcggagctggcccagcgtatgggcaaacgcgcgtttttgattgacgatgcgaaagacatccaggaagagtgggtgaaagaggttaaatgcgtcggcgtgactgcgggcgcatcggctccggatattctggtgcagaatgtggtggcacgtttgcagcagctgggcggtggtgaagccattccgctggaaggccgtgaagaaaacattgttttcgaagtgccgaaagagctgcgtgtcgatattcgtgaagtcgat

SEQ ID NO: 11 - protein, 323 aa, Artificial Sequence
  IspHmut14
MTDPALRAQILLANPRGFCAGVDRAISIVENALAIYGAPIYVRHEVVHNRYVVDSLRERGAIFIEQISEVPDGAILIFSAHGVSQAVRNEAKSRDLTVFDATCPLVTKVHMEVARASRRGEESILIGHAGHPEVEGTMGQYSNPEGGMYLVESPDDVWKLTVKNEEKLSFMTQTTLSVDDTSDVIDALRKRFPKIVGPRKDDICYATTNRLEAVRALAEQAEVVLVVGSKNSSNSNRLAELAQRMGKRAFLIDDAKDIQEEWVKEVKCVGVTAGASAPDILVQNVVARLQQLGGGEAIPLEGREEDIVFEVPKELRVDIREVD

SEQ ID NO: 12 - DNA, 969 bp, Artificial Sequence
  IspHmut14
atgacggatccggccctgagggcccagatcctgttggccaacccgcgtggtttttgtgccggggtagaccgcgctatcagcattgttgaaaacgcgctggccatttacggcgcaccgatatatgtccgtcacgaagtggtacataaccgctatgtggtcgatagcttgcgtgagcgtggggctatctttattgagcagattagcgaagtaccggacggcgcgatcctgattttctccgcacacggtgtttctcaggcggtacgtaacgaagcaaaaagtcgcgatttgacggtgtttgatgccacctgtccgctggtgaccaaagtgcatatggaagtcgcccgcgccagtcgccgtggcgaagaatctattctcatcggtcacgccgggcacccggaagtggaagggacaatgggccagtacagtaacccggaagggggaatgtatctggtcgaatcgccggacgatgtgtggaaactgacggtcaaaaacgaagagaagctctcctttatgacccagaccacgctgtcggtggatgacacgtctgatgtgatcgacgcgctgcgtaaacgcttcccgaaaattgtcggtccgcgcaaagatgacatctgctacgccacgactaaccgtctggaagcggtacgcgccctggcagaacgggcggaagttgtgttggtggtcggttcgaaaaactcctccaactccaaccgtctggcggagctggcccagcgtatgggcaaacgcgcgtttttgattgacgatgcgaaagacatccaggaagagtgggtgaaagaggttaaatgcgtcggcgtgactgcgggcgcatcggctccggatattctggtgcagaatgtggtggcacgtttgcagcagctgggcggtggtgaagccattccgctggaaggccgtgaagaagacattgttttcgaagtgccgaaagagctgcgtgtcgatattcgtgaagtcgat

SEQ ID NO: 13 - DNA, 5130 bp, Artificial Sequence
  pUCara-(40,000)-GESM53his
gcgcccaatacgcaaaccgcctctccccgcgcgttggccgattcattaatgcagctggcacgacaggtttcccgactggaaagcgccatggttatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttgaattcgaggaggtcggtaatgggggataactctcagcgtaaaaacacacgtcaacacatggaggagagcagcagcaagaggagagaatatctgctggaggaaacgacgcgaaaactgcagagaaacgacaccgaatcggtggagaaactcaagcttatcgacaacatccaacagttgggaatcggctactattttgaggacgccatcaacgccgtactccgctcgcctttctccaccggagaagaagacctcttcaccgctgctctgcgcttccgcttgctccgccacaacggcatcgaaatcagccctgaaatattcctaaaattcaaggacgagaggggaaaattcgacgaatcggacacgctagggttactgagcttgtacgaagcgtcaaatttgggggttgcaggagaagaaatattggaggaggctatggagtttgcggaggctcgcctgagacggtcgctgtcagagccggcggcgccgcttcatggtgaggtggcgcaagcgctagatgtgccgaggcatctgagaatggcgaggttggaagcgagacgattcatcgagcagtatggtaaacagagcgatcatgatggagatcttttggagctggcaattttggattataatcaagttcaggctcaacaccaatccgaactcactgaaataatcaggtggtggaaggagctcggtttggtggataagttgagttttgggcgagacagaccattggagtgctttttgtggaccgtggggctcctcccagagcccaagtattcgagcgttagaatagagttggcgaaagccatctctattctcttagtgatcgatgatattttcgatacctatggagagatggatgacctcatcctcttcaccgatgcaattcgaagatgggatcttgaagcaatggaggggctccctgagtacatgaaaatatgctacatggcgttgtacaataccaccaatgaagtatgctacaaagtgctcagggatactggacggattgtcctccttaacctcaaatctacgtggatagacatgattgaaggtttcatggaggaagcaaaatggttcaatggtggaagtgcaccaaaattggaagagtatatagagaatggagtgtccacggcaggagcatacatggcttttgcacacatcttctttctcataggagaaggtgttacacaccaaaattcccaactcttcacccaaaaaccctaccccaaggtcttctccgccgccggccgcattcttcgcctctgggatgatctcggaaccgccaaggaagagcaagagcgaggagatctggcttcgtgcgtgcagttatttatgaaagagaagtcgttgacggaagaggaggcaagaagtcgcattttggaagagataaaaggattatggagggatctgaatggggaactggtctacaacaagaatttgccgttatccataatcaaagtcgcacttaacatggcgagagcttctcaagttgtgtacaagcacgatcaagacacttatttttcaagcgtagacaattatgtggatgccctcttcttcactcaaccatggcatcatcatcatcatcattaactcgaggggccctgatgcggtattttctccttacgcatctgtgcggtatttcacaccgcatatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 14 - DNA, 5039 bp, Artificial Sequence
  pACmod-Plac-idi
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaaggaggattacaaaatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataactcgaggggcccggcgcctgatgcggtattttctccttacgcatctgtgcggtatttcacatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 15 - DNA, 4245 bp, Artificial Sequence
  pAC-mod
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 16 - DNA, 5717 bp, Artificial Sequence
  p15A-dxs
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaggaggccatacatgagaggatctcaccatcaccatcaccatacggatccggccctgagggccagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataactcgaggtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 17 - DNA, 5056 bp, Artificial Sequence
  p15A-dxr
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaggaggccatacatgagaggatctcaccatcaccatcaccatacggatccggccctgagggccaagcaactcaccattctgggctcgaccggctcgattggttgcagcacgctggacgtggtgcgccataatcccgaacacttccgcgtagttgcgctggtggcaggcaaaaatgtcactcgcatggtagaacagtgcctggaattctctccccgctatgccgtaatggacgatgaagcgagtgcgaaacttcttaaaacgatgctacagcaacagggtagccgcaccgaagtcttaagtgggcaacaagccgcttgcgatatggcagcgcttgaggatgttgatcaggtgatggcagccattgttggcgctgctgggctgttacctacgcttgctgcgatccgcgcgggtaaaaccattttgctggccaataaagaatcactggttacctgcggacgtctgtttatggacgccgtaaagcagagcaaagcgcaattgttaccggtcgatagcgaacataacgccatttttcagagtttaccgcaacctatccagcataatctgggatacgctgaccttgagcaaaatggcgtggtgtccattttacttaccgggtctggtggccctttccgtgagacgccattgcgcgatttggcaacaatgacgccggatcaagcctgccgtcatccgaactggtcgatggggcgtaaaatttctgtcgattcggctaccatgatgaacaaaggtctggaatacattgaagcgcgttggctgtttaacgccagcgccagccagatggaagtgctgattcacccgcagtcagtgattcactcaatggtgcgctatcaggacggcagtgttctggcgcagctgggggaaccggatatgcgtacgccaattgcccacaccatggcatggccgaatcgcgtgaactctggcgtgaagccgctcgatttttgcaaactaagtgcgttgacatttgccgcaccggattatgatcgttatccatgcctgaaactggcgatggaggcgttcgaacaaggccaggcagcgacgacagcattgaatgccgcaaacgaaatcaccgttgctgcttttcttgcgcaacaaatccgctttacggatatcgctgcgttgaatttatccgtactggaaaaaatggatatgcgcgaaccacaatgtgtggacgatgtgttatctgttgatgcgaacgcgcgtgaagtcgccagaaaagaggtgatgcgtctcgcaagctaactcgaggggcccgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 18 - DNA, 4564 bp, Artificial Sequence
  p15A-ispD
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaggaggccatacatgagaggatctcaccatcaccatcaccatacggatccggccctgagggccgcaaccactcatttggatgtttgcgccgtggttccggcggccggatttggccgtcgaatgcaaacggaatgtcctaagcaatatctctcaatcggtaatcaaaccattcttgaacactcggtgcatgcgctgctggcgcatccccgggtgaaacgtgtcgtcattgccataagtcctggcgatagccgttttgcacaacttcctctggcgaatcatccgcaaatcaccgttgtagatggcggtgatgagcgtgccgattccgtgctggcaggtctgaaagccgctggcgacgcgcagtgggtattggtgcatgacgccgctcgtccttgtttgcatcaggatgacctcgcgcgattgttggcgttgagcgaaaccagccgcacgggggggatcctcgccgcaccagtgcgcgatactatgaaacgtgccgaaccgggcaaaaatgccattgctcataccgttgatcgcaacggcttatggcacgcgctgacgccgcaatttttccctcgtgagctgttacatgactgtctgacgcgcgctctaaatgaaggcgcgactattaccgacgaagcctcggcgctggaatattgcggattccatcctcagttggtcgaaggccgtgcggataacattaaagtcacgcgcccggaagatttggcactggccgagttttacctcacccgaaccatccatcaggagaatacataactcgaggtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 19 - DNA, 4705 bp, Artificial Sequence
  p15A-ispE
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaggaggccatacatgagaggatctcaccatcaccatcaccatacggatccggccctgagggcccggacacagtggccctctccggcaaaacttaatctgtttttatacattaccggtcagcgtgcggatggttaccacacgctgcaaacgctgtttcagtttcttgattacggcgacaccatcagcattgagcttcgtgacgatggggatattcgtctgttaacgcccgttgaaggcgtggaacatgaagataacctgatcgttcgcgcagcgcgattgttgatgaaaactgcggcagacagcgggcgtcttccgacgggaagcggtgcgaatatcagcattgacaagcgtttgccgatgggcggcggtctcggcggtggttcatccaatgccgcgacggtcctggtggcattaaatcatctctggcaatgcgggctaagcatggatgagctggcggaaatggggctgacgctgggcgcagatgttcctgtctttgttcgggggcatgccgcgtttgccgaaggcgttggtgaaatactaacgccggtggatccgccagagaagtggtatctggtggcgcaccctggtgtaagtattccgactccggtgatttttaaagatcctgaactcccgcgcaatacgccaaaaaggtcaatagaaacgttgctaaaatgtgaattcagcaatgattgcgaggttatcgcaagaaaacgttttcgcgaggttgatgcggtgctttcctggctgttagaatacgccccgtcgcgcctgactgggacaggggcctgtgtctttgctgaatttgatacagagtctgaagcccgccaggtgctagagcaagccccggaatggctcaatggctttgtggcgaaaggcgctaatctttccccattgcacagagccatgctttaactcgaggtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 20 - DNA, 4339 bp, Artificial Sequence
  p15A-ispF
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaggaggccatacatgagaggatctcaccatcaccatcaccatacggatccggccctgagggcccgaattggacacggttttgacgtacatgcctttggcggtgaaggcccaattatcattggtggcgtacgcattccttacgaaaaaggattgctggcgcattctgatggcgacgtggcgctccatgcgttgaccgatgcattgcttggcgcggcggcgctgggggatatcggcaagctgttcccggataccgatccggcatttaaaggtgccgatagccgcgagctgctacgcgaagcctggcgtcgtattcaggcgaagggttatacccttggcaacgtcgatgtcactatcatcgctcaggcaccgaagatgttgccgcacattccacaaatgcgcgtgtttattgccgaagatctcggctgccatatggatgatgttaacgtgaaagccactactacggaaaaactgggatttaccggacgtggggaagggattgcctgtgaagcggtggcgctactcattaaggcaacaaaataactcgaggggcccgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 21 - DNA, 4972 bp, Artificial Sequence
  p15A-ispG
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaggaggccatacatgagaggatctcaccatcaccatcaccatacggatccggccctgagggcccataaccaggctccaattcaacgtagaaaatcaacacgtatttacgttgggaatgtgccgattggcgatggtgctcccatcgccgtacagtccatgaccaatacgcgtacgacagacgtcgaagcaacggtcaatcaaatcaaggcgctggaacgcgttggcgctgatatcgtccgtgtatccgtaccgacgatggacgcggcagaagcgttcaaactcatcaaacagcaggttaacgtgccgctggtggctgacatccacttcgactatcgcattgcgctgaaagtagcggaatacggcgtcgattgtctgcgtattaaccctggcaatatcggtaatgaagagcgtattcgcatggtggttgactgtgcgcgcgataaaaacattccgatccgtattggcgttaacgccggatcgctggaaaaagatctgcaagaaaagtatggcgaaccgacgccgcaggcgttgctggaatctgccatgcgtcatgttgatcatctcgatcgcctgaacttcgatcagttcaaagtcagcgtgaaagcgtctgacgtcttcctcgctgttgagtcttatcgtttgctggcaaaacagatcgatcagccgttgcatctggggatcaccgaagccggtggtgcgcgcagcggggcagtaaaatccgccattggtttaggtctgctgctgtctgaaggcatcggcgacacgctgcgcgtatcgctggcggccgatccggtcgaagagatcaaagtcggtttcgatattttgaaatcgctgcgtatccgttcgcgagggatcaacttcatcgcctgcccgacctgttcgcgtcaggaatttgatgttatcggtacggttaacgcgctggagcaacgcctggaagatatcatcactccgatggacgtttcgattatcggctgcgtggtgaatggcccaggtgaggcgctggtttctacactcggcgtcaccggcggcaacaagaaaagcggcctctatgaagatggcgtgcgcaaagaccgtctggacaacaacgatatgatcgaccagctggaagcacgcattcgtgcgaaagccagtcagctggacgaagcgcgtcgaattgacgttcagcaggttgaaaaataactcgaggtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 22 - DNA, 4804 bp, Artificial Sequence
  p15A-ispHwt
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaggaggccatacatgagaggatctcaccatcaccatcaccatacggatccggccctgagggcccagatcctgttggccaacccgcgtggtttttgtgccggggtagaccgcgctatcagcattgttgaaaacgcgctggccatttacggcgcaccgatatatgtccgtcacgaagtggtacataaccgctatgtggtcgatagcttgcgtgagcgtggggctatctttattgagcagattagcgaagtaccggacggcgcgatcctgattttctccgcacacggtgtttctcaggcggtacgtaacgaagcaaaaagtcgcgatttgacggtgtttgatgccacctgtccgctggtgaccaaagtgcatatggaagtcgcccgcgccagtcgccgtggcgaagaatctattctcatcggtcacgccgggcacccggaagtggaagggacaatgggccagtacagtaacccggaagggggaatgtatctggtcgaatcgccggacgatgtgtggaaactgacggtcaaaaacgaagagaagctctcctttatgacccagaccacgctgtcggtggatgacacgtctgatgtgatcgacgcgctgcgtaaacgcttcccgaaaattgtcggtccgcgcaaagatgacatctgctacgccacgactaaccgtcaggaagcggtacgcgccctggcagaacaggcggaagttgtgttggtggtcggttcgaaaaactcctccaactccaaccgtctggcggagctggcccagcgtatgggcaaacgcgcgtttttgattgacgatgcgaaagacatccaggaagagtgggtgaaagaggttaaatgcgtcggcgtgactgcgggcgcatcggctccggatattctggtgcagaatgtggtggcacgtttgcagcagctgggcggtggtgaagccattccgctggaaggccgtgaagaaaacattgttttcgaagtgccgaaagagctgcgtgtcgatattcgtgaagtcgattaactcgaggtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 23 - DNA, 4402 bp, Artificial Sequence
  p15A-idi
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaggaggccatacatgagaggatctcaccatcaccatcaccatacggatccggccctgagggcccaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataactcgaggtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 24 - DNA, 4759 bp, Artificial Sequence
  p15A-ispA
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaggaggccatacatgagaggatctcaccatcaccatcaccatacggatccggccctgagggccgactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgttcggcgttagcacaaacacgctggacgcacccgctgccgccgttgagtgtatccacgcttactcattaattcatgatgatttaccggcaatggatgatgacgatctgcgtcgcggtttgccaacctgccatgtgaagtttggcgaagcaaacgcgattctcgctggcgacgctttacaaacgctggcgttctcgattttaagcgatgccgatatgccggaagtgtcggaccgcgacagaatttcgatgatttctgaactggcgagcgccagtggtattgccggaatgtgcggtggtcaggcattagatttagacgcggaaggcaaacacgtacctctggacgcgcttgagcgtattcatcgtcataaaaccggcgcattgattcgcgccgccgttcgccttggtgcattaagcgccggagataaaggacgtcgtgctctgccggtactcgacaagtatgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataactcgaggggcccgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 25 - DNA, 4804 bp, Artificial Sequence; identical to SEQ ID NO: 22
  p15A-ispHmut1

SEQ ID NO: 26 - DNA, 4804 bp, Artificial Sequence; identical to SEQ ID NO: 22
  p15A-ispHmut2

SEQ ID NO: 27 - DNA, 4804 bp, Artificial Sequence; identical to SEQ ID NO: 22
  p15A-ispHmut12

SEQ ID NO: 28 - DNA, 4804 bp, Artificial Sequence; identical to SEQ ID NO: 22
  p15A-ispHmut14

SEQ ID NO: 29 - DNA, 6634 bp, Artificial Sequence
  pJ404-PShis-GPPS
ctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatcatttagaataaattttgtgtcgcccttaattgtgagcggataacaattacgagcttcatgcacagtgaaatcatgaaaaatttatttgctttgtgagcggataacaattataatatgtggaattgtgagcgctcacaattccacaacggtttccctctagaaataattttgtttaacttttaggaggtaaaacatatgcgtagacgtggcgattttcattctaacctgtgggacgacgacttaatccagagcctgagcagcccgtatggtgagccatcctatcgcgagcgtgcggagcgcctgattggtgaggtgaagaactctttcaacagcatgagcaatgaggacggtgagagcatcaccccgctggatgatctgattcaacgcttgtggatggttgacagcgtggagcgtctgggtattgaccgccatttcaagaaagaaatcaagtctgcactggatcacgtttatcgctactggagcgaaaagggtatcggctgcggccgtgagagcgtggtcaccgatctgaatagcaccgcactgggtctgcgtacgttgcgcctgcacggttatgatgtgagcgctgacgtgctgaatcacttcaagaatcaaagcggccagttcgcgtgcaccctgaaacagaccgaagatcagatccgtaccgttttgaacctgtaccgcgcgagcctgatcgccttcccgggtgagaaagtcatggatgaagcggagagcttctccgcaaaatacctgaaagaggccctgcaaaagattccggtgagcagctttagccgtgaaatcggtgacgtcctggagtacggttggcacacgtatctgccgcgtctggaagcgcgtaactatatcgacgtgttcggtcaggacaccgagaactccaagagctatatgaaaacggaaaagctgctggagctggcgaagcttgagttcaatatcttccatgcgttgcagaaacgcgagttggagtacttggttcgttggtggaaaggttcgggctcccctcagatgaccttctgtcgtcatcgtcacgttgagtattacaccctggctagctgcattgcgtttgaacctcaacactctggctttcgtctgggttttgccaaagcctgtcatatcatcactgtcctggatgatatgtacgatacgtttggcacccttgacgaactggagctgtttaccagcgctattaagcgctgggacccgagcgccacggagtgcctgccggagtacatgaagggtgtttacatgattgtctacaataccgtgaatgagatgagccaagaagcagacaaagcgcaaggccgcgataccctgaactattgccgtcaggcgtgggaagagtatattgacgcttacatgcaagaagccaagtggattgcgtcgggtgaagtcccaacctttgaagagtactacgaaaatggtaaggtttccagcggtcaccgtgttagcgcactgcaaccgatcctgacgactgacatcccgttcccggagcacgtcctgaaagaagtggacattccgagccaactgaatgacctggcaagcgcgattctgcgtttgcgtggtgacacgcgttgctatcaggcagaccgcgctcgtggcgaagaagcaagctgtatttcctgttacatgaaagataacccgggcacgacggaagaagatgcgctgaaccatctgaacgccatgatctccgatgtgattaaaggcctgaactgggaactgttgaagccgaatagctctgtcccgattagcgcgaaaaagcatgcattcgatattagccgtgcgtttcactgtggctacaagtatcgcgacggttacagcgttgcgaacatcgagactaaatcactggtaaaacgtaccgtgatcgatccggttaccttgggcagcagccaccaccaccatcaccactaataactcgagttcttaaggaggtaatcccatggaatttgacttcaacaaatacatggactccaaagcgatgacggtaaatgaagcactgaacaaagcgatccctctgcgttatccgcagaaaatctacgaaagcatgcgttacagcctgctggcaggcggcaagcgtgttcgtccggttctgtgtattgccgcatgtgaactggtaggtggtaccgaagaactggcgatcccgaccgcgtgcgcaattgaaatgatccacacgatgtccctgatgcacgatgatctgccgtgtatcgacaacgacgatctgcgtcgcggtaaaccgactaaccacaaaattttcggtgaggataccgcagtgactgctggtaacgcactgcactcttacgccttcgagcatatcgcggtttctacttctaaaaccgttggtgctgaccgcatcctgcgtatggtgtccgagctgggtcgtgctactggctctgaaggtgttatgggtggtcagatggtagacatcgcatccgaaggcgatccgtctatcgacctgcagaccctggaatggattcacatccacaaaaccgcaatgctgctggaatgctccgttgtttgcggtgcaatcattggcggtgccagcgaaatcgtaatcgaacgtgcccgtcgctacgcccgctgtgttggtctgctgttccaggtagttgatgacattctggacgtaactaaaagcagcgacgaactgggtaagactgcgggcaaggacctgatctctgataaagccacctacccaaagctgatgggtctggaaaaggccaaggagttctccgatgaactgctgaaccgtgcgaagggtgaactgtcctgcttcgacccagttaaagccgctccgctgctgggcctggcagactacgtggcatttcgtcagaattaagaattcgccccaagggcgacaccccataattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggtaagcggcgcgccatcgaatggcgcaaaacctttcgcggtatggcatgatagcgcccggaagagagtcaattcagggtggtgaatatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaggacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgccagtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 30 - DNA, 4900 bp, Artificial Sequence
  pUC-crtM-crtN
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaaggaggattacaaaatgacaatgatgaatatgaattttaaatattgtcataaaatcatgaagaaacattcaaaaagcttttcttacgcttttgacttgttaccagaagatcaaagaaaagcggtttgggcaatttatgctgtgtgtcgtaaaattgatgacagtatagatgtttatggcgatattcaatttttaaatcaaataaaagaagatatacaatctattgaaaaatacccatatgaacatcatcactttcaaagtgatcgtagaatcatgatggcgcttcagcatgttgcacaacataaaaatatcgcctttcaatctttttataatctcattgatactgtatataaagatcaacattttacaatgtttgaaacggacgctgaattattcggatattgttatggtgttgctggtacagtaggtgaagtattgacgccgattttaagtgatcatgaaacacatcagacatacgatgtcgcaagaagacttggtgaatcgttgcaattgattaatatattaagagatgtcggtgaagattttgacaatgaacggatatattttagtaagcaacgattaaagcaatatgaagttgatattgctgaagtgtaccaaaatggtgttaataatcattatattgacttatgggaatattatgcagctatcgcagaaaaagattttcaagatgttatggatcaaatcaaagtatttagtattgaagcacaaccaatcatagaattagcagcacgtatatatattgaaatactggacgaagtgagacaggctaactatacattacatgaacgtgtttttgtggataagaggaaaaaggcaaagttgtttcatgaaataaatagtaaatatcatagaatatagctcgagaggaggattacaaaatgaagattgcagtaattggtgcaggtgtcacaggattagcagcggcagcccgtattgcttctcaaggtcatgaagtgacgatatttgaaaaaaataataatgtaggcgggcgtatgaatcaattaaagaaagacggctttacatttgatatgggtcccacaattgtcatgatgccagatgtttataaagatgtttttacagcgtgtggtaaaaattatgaagattatattgaattgagacaattacgttatatttacgatgtgtattttgaccacgatgatcgtataacggtgcctacagatttagctgaattacagcaaatgctagaaagtatagaacctggttcaacgcatggttttatgtcctttttaacggatgtttataaaaaatatgaaattgcacgtcgctatttcttagaaagaacgtatcgcaaaccgagtgacttttataatatgacgtcacttgtgcaaggtgctaagttaaaaacgttaaatcatgcagatcagctaattgaacattatattgataacgaaaagatacaaaagcttttagcgtttcaaacgttatacataggaattgatccaaaacgaggcccgtcactatattcaattattcctatgattgaaatgatgtttggtgtgcattttattaaaggcggtatgtatggcatggctcaagggctagcgcaattaaataaagacttaggcgttaatattgaactaaatgctgaaattgagcaaattattattgatcctaaattcaaacgggccgatgcgataaaagtgaatggtgacataagaaaatttgataaaattttatgtacggctgatttccctagtgttgcggaatcattaatgccagattttgcacctattaaaaagtatccaccacataaaattgcagacttagattactcttgttcagcatttttaatgtatatcggtatagatattgatgtgacagatcaagtgagacttcataatgttattttttcagatgactttagaggcaatattgaagaaatatttgagggacgtttatcatatgatccttctatttatgtgtatgtaccagcggtcgctgataaatcacttgcgccagaaggcaaaactggtatttatgtgctaatgccgacgccggaacttaaaacaggtagcggaatcgattggtcagatgaagctttgacgcaacaaataaaggaaattatttatcgtaaattagcaacgattgaagtatttgaagatataaaatcgcatattgtttcagaaacaatctttacgccaaatgattttgagcaaacgtatcatgcgaaatttggttcggcattcggtttaatgccaactttagcgcaaagtaattattatcgtccacaaaatgtatcgcgagattataaagatttatattttgcaggtgcaagtacgcatccaggtgcaggcgttcctattgtcttaacgagtgcgaaaataactgtagatgaaatgattaaagatattgagcggggcgtataagggagtagtctaagggcccggcgcctgatgcggtattttctccttacgcatctgtgcggtatttcacaccgcatatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 31 - DNA, 6634 bp, Artificial Sequence; identical to SEQ ID NO: 29
  pJ404-PtPSQ457L-GPPS

SEQ ID NO: 32 - DNA, 4804 bp, Artificial Sequence; identical to SEQ ID NO: 22
  p15A-ispHwt

SEQ ID NO: 33 - DNA, 6765 bp, Artificial Sequence
  p15A-ispHwt-dxs
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaggaggccatacatgagaggatctcaccatcaccatcaccatacggatccggccctgagggcccagatcctgttggccaacccgcgtggtttttgtgccggggtagaccgcgctatcagcattgttgaaaacgcgctggccatttacggcgcaccgatatatgtccgtcacgaagtggtacataaccgctatgtggtcgatagcttgcgtgagcgtggggctatctttattgagcagattagcgaagtaccggacggcgcgatcctgattttctccgcacacggtgtttctcaggcggtacgtaacgaagcaaaaagtcgcgatttgacggtgtttgatgccacctgtccgctggtgaccaaagtgcatatggaagtcgcccgcgccagtcgccgtggcgaagaatctattctcatcggtcacgccgggcacccggaagtggaagggacaatgggccagtacagtaacccggaagggggaatgtatctggtcgaatcgccggacgatgtgtggaaactgacggtcaaaaacgaagagaagctctcctttatgacccagaccacgctgtcggtggatgacacgtctgatgtgatcgacgcgctgcgtaaacgcttcccgaaaattgtcggtccgcgcaaagatgacatctgctacgccacgactaaccgtcaggaagcggtacgcgccctggcagaacaggcggaagttgtgttggtggtcggttcgaaaaactcctccaactccaaccgtctggcggagctggcccagcgtatgggcaaacgcgcgtttttgattgacgatgcgaaagacatccaggaagagtgggtgaaagaggttaaatgcgtcggcgtgactgcgggcgcatcggctccggatattctggtgcagaatgtggtggcacgtttgcagcagctgggcggtggtgaagccattccgctggaaggccgtgaagaaaacattgttttcgaagtgccgaaagagctgcgtgtcgatattcgtgaagtcgattaactcgaggtcgaccgatgcccttgagagccttcactagaggaggccatacatgagaggatctcaccatcaccatcaccatacggatccggccctgagggccagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataactcgaggtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 34 - DNA, 7803 bp, Artificial Sequence
  p15A-ispHwt-dxs-ispG
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaggaggccatacatgagaggatctcaccatcaccatcaccatacggatccggccctgagggcccagatcctgttggccaacccgcgtggtttttgtgccggggtagaccgcgctatcagcattgttgaaaacgcgctggccatttacggcgcaccgatatatgtccgtcacgaagtggtacataaccgctatgtggtcgatagcttgcgtgagcgtggggctatctttattgagcagattagcgaagtaccggacggcgcgatcctgattttctccgcacacggtgtttctcaggcggtacgtaacgaagcaaaaagtcgcgatttgacggtgtttgatgccacctgtccgctggtgaccaaagtgcatatggaagtcgcccgcgccagtcgccgtggcgaagaatctattctcatcggtcacgccgggcacccggaagtggaagggacaatgggccagtacagtaacccggaagggggaatgtatctggtcgaatcgccggacgatgtgtggaaactgacggtcaaaaacgaagagaagctctcctttatgacccagaccacgctgtcggtggatgacacgtctgatgtgatcgacgcgctgcgtaaacgcttcccgaaaattgtcggtccgcgcaaagatgacatctgctacgccacgactaaccgtcaggaagcggtacgcgccctggcagaacaggcggaagttgtgttggtggtcggttcgaaaaactcctccaactccaaccgtctggcggagctggcccagcgtatgggcaaacgcgcgtttttgattgacgatgcgaaagacatccaggaagagtgggtgaaagaggttaaatgcgtcggcgtgactgcgggcgcatcggctccggatattctggtgcagaatgtggtggcacgtttgcagcagctgggcggtggtgaagccattccgctggaaggccgtgaagaaaacattgttttcgaagtgccgaaagagctgcgtgtcgatattcgtgaagtcgattaactcgaggtcgaccgatgcccttgagagccttcactagaggaggccatacatgagaggatctcaccatcaccatcaccatacggatccggccctgagggccagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataactcgaggtcgaccgatgcccttgagagccttcaacccagtcagactagaggtataagtaggtaacttcatgagaggatctcaccatcaccatcaccatacggatccggccctgagggcccataaccaggctccaattcaacgtagaaaatcaacacgtatttacgttgggaatgtgccgattggcgatggtgctcccatcgccgtacagtccatgaccaatacgcgtacgacagacgtcgaagcaacggtcaatcaaatcaaggcgctggaacgcgttggcgctgatatcgtccgtgtatccgtaccgacgatggacgcggcagaagcgttcaaactcatcaaacagcaggttaacgtgccgctggtggctgacatccacttcgactatcgcattgcgctgaaagtagcggaatacggcgtcgattgtctgcgtattaaccctggcaatatcggtaatgaagagcgtattcgcatggtggttgactgtgcgcgcgataaaaacattccgatccgtattggcgttaacgccggatcgctggaaaaagatctgcaagaaaagtatggcgaaccgacgccgcaggcgttgctggaatctgccatgcgtcatgttgatcatctcgatcgcctgaacttcgatcagttcaaagtcagcgtgaaagcgtctgacgtcttcctcgctgttgagtcttatcgtttgctggcaaaacagatcgatcagccgttgcatctggggatcaccgaagccggtggtgcgcgcagcggggcagtaaaatccgccattggtttaggtctgctgctgtctgaaggcatcggcgacacgctgcgcgtatcgctggcggccgatccggtcgaagagatcaaagtcggtttcgatattttgaaatcgctgcgtatccgttcgcgagggatcaacttcatcgcctgcccgacctgttcgcgtcaggaatttgatgttatcggtacggttaacgcgctggagcaacgcctggaagatatcatcactccgatggacgtttcgattatcggctgcgtggtgaatggcccaggtgaggcgctggtttctacactcggcgtcaccggcggcaacaagaaaagcggcctctatgaagatggcgtgcgcaaagaccgtctggacaacaacgatatgatcgaccagctggaagcacgcattcgtgcgaaagccagtcagctggacgaagcgcgtcgaattgacgttcagcaggttgaaaaataagcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 35 - DNA, 9093 bp, Artificial Sequence
  p15A-ispHwt-dxs-ispG-dxr
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaggaggccatacatgagaggatctcaccatcaccatcaccatacggatccggccctgagggcccagatcctgttggccaacccgcgtggtttttgtgccggggtagaccgcgctatcagcattgttgaaaacgcgctggccatttacggcgcaccgatatatgtccgtcacgaagtggtacataaccgctatgtggtcgatagcttgcgtgagcgtggggctatctttattgagcagattagcgaagtaccggacggcgcgatcctgattttctccgcacacggtgtttctcaggcggtacgtaacgaagcaaaaagtcgcgatttgacggtgtttgatgccacctgtccgctggtgaccaaagtgcatatggaagtcgcccgcgccagtcgccgtggcgaagaatctattctcatcggtcacgccgggcacccggaagtggaagggacaatgggccagtacagtaacccggaagggggaatgtatctggtcgaatcgccggacgatgtgtggaaactgacggtcaaaaacgaagagaagctctcctttatgacccagaccacgctgtcggtggatgacacgtctgatgtgatcgacgcgctgcgtaaacgcttcccgaaaattgtcggtccgcgcaaagatgacatctgctacgccacgactaaccgtcaggaagcggtacgcgccctggcagaacaggcggaagttgtgttggtggtcggttcgaaaaactcctccaactccaaccgtctggcggagctggcccagcgtatgggcaaacgcgcgtttttgattgacgatgcgaaagacatccaggaagagtgggtgaaagaggttaaatgcgtcggcgtgactgcgggcgcatcggctccggatattctggtgcagaatgtggtggcacgtttgcagcagctgggcggtggtgaagccattccgctggaaggccgtgaagaaaacattgttttcgaagtgccgaaagagctgcgtgtcgatattcgtgaagtcgattaactcgaggtcgaccgatgcccttgagagccttcactagaggaggccatacatgagaggatctcaccatcaccatcaccatacggatccggccctgagggccagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataactcgaggtcgaccgatgcccttgagagccttcaacccagtcagactagaggtataagtaggtaacttcatgagaggatctcaccatcaccatcaccatacggatccggccctgagggcccataaccaggctccaattcaacgtagaaaatcaacacgtatttacgttgggaatgtgccgattggcgatggtgctcccatcgccgtacagtccatgaccaatacgcgtacgacagacgtcgaagcaacggtcaatcaaatcaaggcgctggaacgcgttggcgctgatatcgtccgtgtatccgtaccgacgatggacgcggcagaagcgttcaaactcatcaaacagcaggttaacgtgccgctggtggctgacatccacttcgactatcgcattgcgctgaaagtagcggaatacggcgtcgattgtctgcgtattaaccctggcaatatcggtaatgaagagcgtattcgcatggtggttgactgtgcgcgcgataaaaacattccgatccgtattggcgttaacgccggatcgctggaaaaagatctgcaagaaaagtatggcgaaccgacgccgcaggcgttgctggaatctgccatgcgtcatgttgatcatctcgatcgcctgaacttcgatcagttcaaagtcagcgtgaaagcgtctgacgtcttcctcgctgttgagtcttatcgtttgctggcaaaacagatcgatcagccgttgcatctggggatcaccgaagccggtggtgcgcgcagcggggcagtaaaatccgccattggtttaggtctgctgctgtctgaaggcatcggcgacacgctgcgcgtatcgctggcggccgatccggtcgaagagatcaaagtcggtttcgatattttgaaatcgctgcgtatccgttcgcgagggatcaacttcatcgcctgcccgacctgttcgcgtcaggaatttgatgttatcggtacggttaacgcgctggagcaacgcctggaagatatcatcactccgatggacgtttcgattatcggctgcgtggtgaatggcccaggtgaggcgctggtttctacactcggcgtcaccggcggcaacaagaaaagcggcctctatgaagatggcgtgcgcaaagaccgtctggacaacaacgatatgatcgaccagctggaagcacgcattcgtgcgaaagccagtcagctggacgaagcgcgtcgaattgacgttcagcaggttgaaaaataacgctaccagcgaaggatacaataaaacaactaggaagagtcattaatgagaggatctcaccatcaccatcaccatacggatccggccctgagggccaagcaactcaccattctgggctcgaccggctcgattggttgcagcacgctggacgtggtgcgccataatcccgaacacttccgcgtagttgcgctggtggcaggcaaaaatgtcactcgcatggtagaacagtgcctggaattctctccccgctatgccgtaatggacgatgaagcgagtgcgaaacttcttaaaacgatgctacagcaacagggtagccgcaccgaagtcttaagtgggcaacaagccgcttgcgatatggcagcgcttgaggatgttgatcaggtgatggcagccattgttggcgctgctgggctgttacctacgcttgctgcgatccgcgcgggtaaaaccattttgctggccaataaagaatcactggttacctgcggacgtctgtttatggacgccgtaaagcagagcaaagcgcaattgttaccggtcgatagcgaacataacgccatttttcagagtttaccgcaacctatccagcataatctgggatacgctgaccttgagcaaaatggcgtggtgtccattttacttaccgggtctggtggccctttccgtgagacgccattgcgcgatttggcaacaatgacgccggatcaagcctgccgtcatccgaactggtcgatggggcgtaaaatttctgtcgattcggctaccatgatgaacaaaggtctggaatacattgaagcgcgttggctgtttaacgccagcgccagccagatggaagtgctgattcacccgcagtcagtgattcactcaatggtgcgctatcaggacggcagtgttctggcgcagctgggggaaccggatatgcgtacgccaattgcccacaccatggcatggccgaatcgcgtgaactctggcgtgaagccgctcgatttttgcaaactaagtgcgttgacatttgccgcaccggattatgatcgttatccatgcctgaaactggcgatggaggcgttcgaacaaggccaggcagcgacgacagcattgaatgccgcaaacgaaatcaccgttgctgcttttcttgcgcaacaaatccgctttacggatatcgctgcgttgaatttatccgtactggaaaaaatggatatgcgcgaaccacaatgtgtggacgatgtgttatctgttgatgcgaacgcgcgtgaagtcgccagaaaagaggtgatgcgtctcgcaagctaagcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 36 - DNA, 4804 bp, Artificial Sequence
  p15A-Plac/lacO-ispHmut12
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaggaggccatacatgagaggatctcaccatcaccatcaccatacggatccggccctgagggcccagatcctgttggccaacccgcgtggtttttgtgccggggtagaccgcgctatcagcattgttgaaaacgcgctggccatttacggcgcaccgatatatgtccgtcacgaagtggtacataaccgctatgtggtcgatagcttgcgtgagcgtggggctatctttattgagcagattagcgaagtaccggacggcgcgatcctgattttctccgcacacggtgtttctcaggcggtacgtaacgaagcaaaaagtcgcgatttgacggtgtttgatgccacctgtccgcaggtgaccaaagtgcatatggaagtcgcccgcgccagtcgccgtggcgaagaatctattctcatcggtcacgccgggcacccggaagtggaagggacaatgggccagtacagtaacccggaagggggaatgtatctggtcgaatcgccggacgatgtgtggaaactgacggtcaaaaacgaagagaagctctcctttatgacccagaccacgctgtcggtggatgacacgtctgatgtgatcgacgcgctgcgtaaacgcttcccgaaaattgtcggtccgcgcaaagatgacatctgctacgccacgactaaccgtcaggaagcggtacgcgccctggcagaacaggcggaagttgtgttggtggtcggttcgaaaaactcctccaactccaaccgtctggcggagctggcccagcgtatgggcaaacgcgcgtttttgattgacgatgcgaaagacatccaggaagagtgggtgaaagaggttaaatgcgtcggcgtgactgcgggcgcatcggctccggatattctggtgcagaatgtggtggcacgtttgcagcagctgggcggtggtgaagccattccgctggaaggccgtgaagaaaacattgttttcgaagtgccgaaagagctgcgtgtcgatattcgtgaagtcgattaactcgaggtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 37 - DNA, 6765 bp, Artificial Sequence
  p15A- ispHmut12-dxs
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaggaggccatacatgagaggatctcaccatcaccatcaccatacggatccggccctgagggcccagatcctgttggccaacccgcgtggtttttgtgccggggtagaccgcgctatcagcattgttgaaaacgcgctggccatttacggcgcaccgatatatgtccgtcacgaagtggtacataaccgctatgtggtcgatagcttgcgtgagcgtggggctatctttattgagcagattagcgaagtaccggacggcgcgatcctgattttctccgcacacggtgtttctcaggcggtacgtaacgaagcaaaaagtcgcgatttgacggtgtttgatgccacctgtccgcaggtgaccaaagtgcatatggaagtcgcccgcgccagtcgccgtggcgaagaatctattctcatcggtcacgccgggcacccggaagtggaagggacaatgggccagtacagtaacccggaagggggaatgtatctggtcgaatcgccggacgatgtgtggaaactgacggtcaaaaacgaagagaagctctcctttatgacccagaccacgctgtcggtggatgacacgtctgatgtgatcgacgcgctgcgtaaacgcttcccgaaaattgtcggtccgcgcaaagatgacatctgctacgccacgactaaccgtcaggaagcggtacgcgccctggcagaacaggcggaagttgtgttggtggtcggttcgaaaaactcctccaactccaaccgtctggcggagctggcccagcgtatgggcaaacgcgcgtttttgattgacgatgcgaaagacatccaggaagagtgggtgaaagaggttaaatgcgtcggcgtgactgcgggcgcatcggctccggatattctggtgcagaatgtggtggcacgtttgcagcagctgggcggtggtgaagccattccgctggaaggccgtgaagaaaacattgttttcgaagtgccgaaagagctgcgtgtcgatattcgtgaagtcgattaactcgaggtcgaccgatgcccttgagagccttcactagaggaggccatacatgagaggatctcaccatcaccatcaccatacggatccggccctgagggccagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataactcgaggtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 38 - DNA, 7803 bp, Artificial Sequence
  p15A-ispHmut12-dxs-ispG
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaggaggccatacatgagaggatctcaccatcaccatcaccatacggatccggccctgagggcccagatcctgttggccaacccgcgtggtttttgtgccggggtagaccgcgctatcagcattgttgaaaacgcgctggccatttacggcgcaccgatatatgtccgtcacgaagtggtacataaccgctatgtggtcgatagcttgcgtgagcgtggggctatctttattgagcagattagcgaagtaccggacggcgcgatcctgattttctccgcacacggtgtttctcaggcggtacgtaacgaagcaaaaagtcgcgatttgacggtgtttgatgccacctgtccgcaggtgaccaaagtgcatatggaagtcgcccgcgccagtcgccgtggcgaagaatctattctcatcggtcacgccgggcacccggaagtggaagggacaatgggccagtacagtaacccggaagggggaatgtatctggtcgaatcgccggacgatgtgtggaaactgacggtcaaaaacgaagagaagctctcctttatgacccagaccacgctgtcggtggatgacacgtctgatgtgatcgacgcgctgcgtaaacgcttcccgaaaattgtcggtccgcgcaaagatgacatctgctacgccacgactaaccgtcaggaagcggtacgcgccctggcagaacaggcggaagttgtgttggtggtcggttcgaaaaactcctccaactccaaccgtctggcggagctggcccagcgtatgggcaaacgcgcgtttttgattgacgatgcgaaagacatccaggaagagtgggtgaaagaggttaaatgcgtcggcgtgactgcgggcgcatcggctccggatattctggtgcagaatgtggtggcacgtttgcagcagctgggcggtggtgaagccattccgctggaaggccgtgaagaaaacattgttttcgaagtgccgaaagagctgcgtgtcgatattcgtgaagtcgattaactcgaggtcgaccgatgcccttgagagccttcactagaggaggccatacatgagaggatctcaccatcaccatcaccatacggatccggccctgagggccagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataactcgaggtcgaccgatgcccttgagagccttcaacccagtcagactagaggtataagtaggtaacttcatgagaggatctcaccatcaccatcaccatacggatccggccctgagggcccataaccaggctccaattcaacgtagaaaatcaacacgtatttacgttgggaatgtgccgattggcgatggtgctcccatcgccgtacagtccatgaccaatacgcgtacgacagacgtcgaagcaacggtcaatcaaatcaaggcgctggaacgcgttggcgctgatatcgtccgtgtatccgtaccgacgatggacgcggcagaagcgttcaaactcatcaaacagcaggttaacgtgccgctggtggctgacatccacttcgactatcgcattgcgctgaaagtagcggaatacggcgtcgattgtctgcgtattaaccctggcaatatcggtaatgaagagcgtattcgcatggtggttgactgtgcgcgcgataaaaacattccgatccgtattggcgttaacgccggatcgctggaaaaagatctgcaagaaaagtatggcgaaccgacgccgcaggcgttgctggaatctgccatgcgtcatgttgatcatctcgatcgcctgaacttcgatcagttcaaagtcagcgtgaaagcgtctgacgtcttcctcgctgttgagtcttatcgtttgctggcaaaacagatcgatcagccgttgcatctggggatcaccgaagccggtggtgcgcgcagcggggcagtaaaatccgccattggtttaggtctgctgctgtctgaaggcatcggcgacacgctgcgcgtatcgctggcggccgatccggtcgaagagatcaaagtcggtttcgatattttgaaatcgctgcgtatccgttcgcgagggatcaacttcatcgcctgcccgacctgttcgcgtcaggaatttgatgttatcggtacggttaacgcgctggagcaacgcctggaagatatcatcactccgatggacgtttcgattatcggctgcgtggtgaatggcccaggtgaggcgctggtttctacactcggcgtcaccggcggcaacaagaaaagcggcctctatgaagatggcgtgcgcaaagaccgtctggacaacaacgatatgatcgaccagctggaagcacgcattcgtgcgaaagccagtcagctggacgaagcgcgtcgaattgacgttcagcaggttgaaaaataagcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 39 - DNA, 9093 bp, Artificial Sequence
  p15A-ispHmut12-dxs-ispG-dxr
  misc_feature (4710)..(4710): n is a, c, g, or t
  misc_feature (4717)..(4717): n is a, c, g, or t
  misc_feature (4720)..(4720): n is a, c, g, or t
  misc_feature (4722)..(4722): n is a, c, g, or t
  misc_feature (4724)..(4724): n is a, c, g, or t
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttcaagatttcagtgcaatttatctcttcaaatgtagcacctgaagtcagccccatacgatataagttgtaattctcatgtttgacagcttatcatcgataggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacacatatggaattctctagaggaggccatacatgagaggatctcaccatcaccatcaccatacggatccggccctgagggcccagatcctgttggccaacccgcgtggtttttgtgccggggtagaccgcgctatcagcattgttgaaaacgcgctggccatttacggcgcaccgatatatgtccgtcacgaagtggtacataaccgctatgtggtcgatagcttgcgtgagcgtggggctatctttattgagcagattagcgaagtaccggacggcgcgatcctgattttctccgcacacggtgtttctcaggcggtacgtaacgaagcaaaaagtcgcgatttgacggtgtttgatgccacctgtccgcaggtgaccaaagtgcatatggaagtcgcccgcgccagtcgccgtggcgaagaatctattctcatcggtcacgccgggcacccggaagtggaagggacaatgggccagtacagtaacccggaagggggaatgtatctggtcgaatcgccggacgatgtgtggaaactgacggtcaaaaacgaagagaagctctcctttatgacccagaccacgctgtcggtggatgacacgtctgatgtgatcgacgcgctgcgtaaacgcttcccgaaaattgtcggtccgcgcaaagatgacatctgctacgccacgactaaccgtcaggaagcggtacgcgccctggcagaacaggcggaagttgtgttggtggtcggttcgaaaaactcctccaactccaaccgtctggcggagctggcccagcgtatgggcaaacgcgcgtttttgattgacgatgcgaaagacatccaggaagagtgggtgaaagaggttaaatgcgtcggcgtgactgcgggcgcatcggctccggatattctggtgcagaatgtggtggcacgtttgcagcagctgggcggtggtgaagccattccgctggaaggccgtgaagaaaacattgttttcgaagtgccgaaagagctgcgtgtcgatattcgtgaagtcgattaactcgaggtcgaccgatgcccttgagagccttcactagaggaggccatacatgagaggatctcaccatcaccatcaccatacggatccggccctgagggccagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataactcgaggtcgaccgatgcccttgagagccttcaacccagtcagactagagntataagnagntnanttcatgagaggatctcaccatcaccatcaccatacggatccggccctgagggcccataaccaggctccaattcaacgtagaaaatcaacacgtatttacgttgggaatgtgccgattggcgatggtgctcccatcgccgtacagtccatgaccaatacgcgtacgacagacgtcgaagcaacggtcaatcaaatcaaggcgctggaacgcgttggcgctgatatcgtccgtgtatccgtaccgacgatggacgcggcagaagcgttcaaactcatcaaacagcaggttaacgtgccgctggtggctgacatccacttcgactatcgcattgcgctgaaagtagcggaatacggcgtcgattgtctgcgtattaaccctggcaatatcggtaatgaagagcgtattcgcatggtggttgactgtgcgcgcgataaaaacattccgatccgtattggcgttaacgccggatcgctggaaaaagatctgcaagaaaagtatggcgaaccgacgccgcaggcgttgctggaatctgccatgcgtcatgttgatcatctcgatcgcctgaacttcgatcagttcaaagtcagcgtgaaagcgtctgacgtcttcctcgctgttgagtcttatcgtttgctggcaaaacagatcgatcagccgttgcatctggggatcaccgaagccggtggtgcgcgcagcggggcagtaaaatccgccattggtttaggtctgctgctgtctgaaggcatcggcgacacgctgcgcgtatcgctggcggccgatccggtcgaagagatcaaagtcggtttcgatattttgaaatcgctgcgtatccgttcgcgagggatcaacttcatcgcctgcccgacctgttcgcgtcaggaatttgatgttatcggtacggttaacgcgctggagcaacgcctggaagatatcatcactccgatggacgtttcgattatcggctgcgtggtgaatggcccaggtgaggcgctggtttctacactcggcgtcaccggcggcaacaagaaaagcggcctctatgaagatggcgtgcgcaaagaccgtctggacaacaacgatatgatcgaccagctggaagcacgcattcgtgcgaaagccagtcagctggacgaagcgcgtcgaattgacgttcagcaggttgaaaaataacgctaccagcgaaggatacaataaaacaactaggaagagtcattaatgagaggatctcaccatcaccatcaccatacggatccggccctgagggccaagcaactcaccattctgggctcgaccggctcgattggttgcagcacgctggacgtggtgcgccataatcccgaacacttccgcgtagttgcgctggtggcaggcaaaaatgtcactcgcatggtagaacagtgcctggaattctctccccgctatgccgtaatggacgatgaagcgagtgcgaaacttcttaaaacgatgctacagcaacagggtagccgcaccgaagtcttaagtgggcaacaagccgcttgcgatatggcagcgcttgaggatgttgatcaggtgatggcagccattgttggcgctgctgggctgttacctacgcttgctgcgatccgcgcgggtaaaaccattttgctggccaataaagaatcactggttacctgcggacgtctgtttatggacgccgtaaagcagagcaaagcgcaattgttaccggtcgatagcgaacataacgccatttttcagagtttaccgcaacctatccagcataatctgggatacgctgaccttgagcaaaatggcgtggtgtccattttacttaccgggtctggtggccctttccgtgagacgccattgcgcgatttggcaacaatgacgccggatcaagcctgccgtcatccgaactggtcgatggggcgtaaaatttctgtcgattcggctaccatgatgaacaaaggtctggaatacattgaagcgcgttggctgtttaacgccagcgccagccagatggaagtgctgattcacccgcagtcagtgattcactcaatggtgcgctatcaggacggcagtgttctggcgcagctgggggaaccggatatgcgtacgccaattgcccacaccatggcatggccgaatcgcgtgaactctggcgtgaagccgctcgatttttgcaaactaagtgcgttgacatttgccgcaccggattatgatcgttatccatgcctgaaactggcgatggaggcgttcgaacaaggccaggcagcgacgacagcattgaatgccgcaaacgaaatcaccgttgctgcttttcttgcgcaacaaatccgctttacggatatcgctgcgttgaatttatccgtactggaaaaaatggatatgcgcgaaccacaatgtgtggacgatgtgttatctgttgatgcgaacgcgcgtgaagtcgccagaaaagaggtgatgcgtctcgcaagctaagcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg